SEQUENCE LISTING (7 sequences)

SEQ ID NO: 1 - DNA, 2394 bp, Fragaria sp.
  source 1..2394: mol_type = other DNA
atggccttacatgggagaatgcttcttttgcaactctctttggccgcagtgctattagcaacgacaatgctagcagctgctcaagcctcaccgcctgtctgcaatgagagctgcggtggtgtctcagttccatatccatttggtctcactgatggttgttacctacatgtgccgggacaagattctcagcagccattcaagatcacttgcaacaccaccacctcgcaaccatccctacagttctcggacagcgataacttccccacaaatattgccaacatttttttggaagagagtgcgctgcaagtaatgatggccccgtcctataactgctacaccaacaatacatacaacccctccatggacaaagtcatggacctcaatcttccgccttcttacaccctctccaacagaaacaaggtttataaccttgggtgcaacaaagtaacgaaactcataggttatcctcttgaagttacagaccctgagcagctctttcaagtacggttcagcggtgtaagcttgtgccaagatgaattcggcaagagattccccgacacttgcaccggattcgggtgctccgtaaattctatccctagcggactgcagaatattacggtggatgtgtggacactcggtaccggcctcggaaaaacggagcagtggggactgagctacccttgcagttacggcttcgttgtggatgaacgcaacttcacattcgccgggaatcaaagttttgttgcactgaggtccggcgcaataaagaagcttccggtgcttgctaattgggcgattgggaatgacaactgcgaagcagcaaagaagaagaacgagactgcttttgcatgcaagactgtgaattccaagtgtgtcgaccgtgctggtggtggttacttttgccagtgtgaggatggttacgaagggaacccatacctccccgataattgcctagatatcaatgagtgcaagcattcaaccctttgcagtggtcctgcaacttgcataaactcaattggaagttacacctgtaaatgtcacaagggccatagaaatgatgaccacgacaagaataagtgtgtcctaatcactgaaacctccagcaaaaatgacaaagaaatgaaaatttccctgggtgtctctttgaccttcctagtcatactgattataactttttcgatatactgcgtactgaaaagaagaaagttcaagcatctctgtgacaggtactacaaagacaatgggggcttcttgttaccacaagaaatgcagaagtacaaagggtctcaggcacccagaatctttaaattagaagagcttaacaaggcaaccaacaagtttgatccccatgaaataattggagaaggaggctttggattggtttacaagggaacattgccggacaacaagcaggaggttgccataaagaagtcaaaaactgatgctcccaccatgacccccgaaagtaggatcgcccacactaagcagttcattaatgagatgattgttctctctggaatcaagcatagaaatgtcgtgaggctcttaggttgttgtttggagaccaaaacgcctatattagtgtacgagtttgtctgcaatggcacactttatgagcacattcataaacagaaaggcaaaggacctctttcctttaggcaacgagtgaagatagcagcagaaacagcaggatccctagcatacttgcactacaacgcttcttccacgccaattctgcatcgagacgtaaaggcatctaacatcctactggatgagaattgcacagccaaaatatcagactttggagcttcaaaattggttcccgaagatgaaaacactcaactggctactttggtgcaagggacgctagggtacttggatcctcaatatattcaaacacacacactgacggagaagagtgatgtctacagtttcggggttgtccttgtggaactgataacgagtcaagcggcaattaattctaacaagaagcttgaggcagagaaaaacctagccaattgttttttaaagtcggtcgaagataatcacttggatcagattcttgatcatgaaattatcaaagatagcttagaaatagctgaacaagtagcccatctcgccaaaagatgtttgagtttgaaaggagatgataggcctgccatgagagaagtagaaacggagctagtggcaatattggcagttatggaaaagcgtcccggaggaaagcctgactcctcccccaaagagaccgattacttgcttgcagcgtcaccttcaaatgctttcgttgttgatgttagaagtgatgaaggtgaactcacaaccagcatagattatgaccagagcatgcagaatcaatcccagatgacgaggccttatgatagtgggagatag

SEQ ID NO: 2 - protein, 797 aa, Fragaria sp.
MALHGRMLLLQLSLAAVLLATTMLAAAQASPPVCNESCGGVSVPYPFGLTDGCYLHVPGQDSQQPFKITCNTTTSQPSLQFSDSDNFPTNIANIFLEESALQVMMAPSYNCYTNNTYNPSMDKVMDLNLPPSYTLSNRNKVYNLGCNKVTKLIGYPLEVTDPEQLFQVRFSGVSLCQDEFGKRFPDTCTGFGCSVNSIPSGLQNITVDVWTLGTGLGKTEQWGLSYPCSYGFVVDERNFTFAGNQSFVALRSGAIKKLPVLANWAIGNDNCEAAKKKNETAFACKTVNSKCVDRAGGGYFCQCEDGYEGNPYLPDNCLDINECKHSTLCSGPATCINSIGSYTCKCHKGHRNDDHDKNKCVLITETSSKNDKEMKISLGVSLTFLVILIITFSIYCVLKRRKFKHLCDRYYKDNGGFLLPQEMQKYKGSQAPRIFKLEELNKATNKFDPHEIIGEGGFGLVYKGTLPDNKQEVAIKKSKTDAPTMTPESRIAHTKQFINEMIVLSGIKHRNVVRLLGCCLETKTPILVYEFVCNGTLYEHIHKQKGKGPLSFRQRVKIAAETAGSLAYLHYNASSTPILHRDVKASNILLDENCTAKISDFGASKLVPEDENTQLATLVQGTLGYLDPQYIQTHTLTEKSDVYSFGVVLVELITSQAAINSNKKLEAEKNLANCFLKSVEDNHLDQILDHEIIKDSLEIAEQVAHLAKRCLSLKGDDRPAMREVETELVAILAVMEKRPGGKPDSSPKETDYLLAASPSNAFVVDVRSDEGELTTSIDYDQSMQNQSQMTRPYDSGR

SEQ ID NO: 3 - DNA, 1947 bp, Fragaria sp.
  source 1..1947: mol_type = other DNA
atggctaaacatcagggaccaaacaatgaaatccaaatgagcattacaccagatgtgtcttatgatttccatagtcctgaggctctaaaaccgacacgcgaacatggacaacctcaagcaagcagcaaaagatggaattggccagcagttgccggagtttcttcagctttgaacccatggtggaataagatactgataacttcatgtgtgattgcagttttttttgatcccttgttcttttacattccatacaccagcgaggaaaaaaagtgtatgggaaacgacgaaagagtgcagactccagctctgattttccgatcagtcacagacatcattttcctggtgcatatcatataccaactatatgtggctatcaaatttgcacactcaagggtttccgaggatgaaagtttggtttttagatatgaatcggtatcccgagagcaaatgattcagtttgcgaaggcattttctcacaagttgtcatggcgatctttcctaactgacgttttcgctattttgcccataccacaacttctaatagtaggattgtttttcaacatcggaggcaataactacttgttaaaaagaaagatcgtgggcggttttcttctaatccaatatgtgccaagaatctatcgaatttatctttcatctgtcgcggtcacagaatctgcattgtgggttagaggtgtatttaacttcttcctctacattcttgcgagtcatgcacttggagccttttggtacttttgttctattcagcgagagacatcatgttggtatcgaacttgcgtcgctacaaatatatcagaatcaaaatgcaccttttactgccataacagtactgctgcacttataaccccacaattcaagacaactctagacgagaaatgcattctaaaagttccatataacatgaccgatccaccatttgattttggaatattttttgatgccctcaaaaatgatatccaagggaaaataaatgttccacaaaagattggttactgtttttggtggggcttgcgaaacttgagtaattttggtacaagtttggaaacaagtacctatctgtgggaaaacagctttgccattttaatttctatcatcggcttgctgttgtttttatacctcattggaaatgtacagatttatatgcagagggcaactaccagagcggaggaggtaagagagaagcttcggatcaaaaagaacgatatagataattgggtccaaaataatggtatcgagaaggacatgaaggaagagatcatgaaaaacatcgttgaaaagttggaaaacgacataagtgcagatctggatgatatatactctattcttcccccgtataccaggaaagttgtgaagcgatttgtcggcatgaggacgctaagaaatgtaccaatgcttagccaggtggatggaagagttttgaagatgatgtgcgactatctgaagccggtcaagtaccaggagaatacaatggtttttgaaacgggagtaccatttgatagaatggtcttcattacagatgggtcaatgtggacttacacgattgctactgatcatcagactagtcattctggaaaagcaacgtcaggaggactagttacttcttcgtccacggaagctcacttcctcaagaagggtgatgcttatggacacatgcttctgcaattagcatcatcatcttctacggcggttcctatctcagttgcaaatgtcaggtgccacacaaaggtagaggcgtttgtcctcatggccaatgacttgagaaacatagtaactgcgtgcggagatttgtggccggcttttgactacaaggcttctcaagatcaggcggctgatcagatggcaccgccggggcattttcagcaacaacaagggccaaagaagcgtactgtagcatcatcaaatatccatcctaccggctacagtgggtga

SEQ ID NO: 4 - protein, 648 aa, Fragaria sp.
MAKHQGPNNEIQMSITPDVSYDFHSPEALKPTREHGQPQASSKRWNWPAVAGVSSALNPWWNKILITSCVIAVFFDPLFFYIPYTSEEKKCMGNDERVQTPALIFRSVTDIIFLVHIIYQLYVAIKFAHSRVSEDESLVFRYESVSREQMIQFAKAFSHKLSWRSFLTDVFAILPIPQLLIVGLFFNIGGNNYLLKRKIVGGFLLIQYVPRIYRIYLSSVAVTESALWVRGVFNFFLYILASHALGAFWYFCSIQRETSCWYRTCVATNISESKCTFYCHNSTAALITPQFKTTLDEKCILKVPYNMTDPPFDFGIFFDALKNDIQGKINVPQKIGYCFWWGLRNLSNFGTSLETSTYLWENSFAILISIIGLLLFLYLIGNVQIYMQRATTRAEEVREKLRIKKNDIDNWVQNNGIEKDMKEEIMKNIVEKLENDISADLDDIYSILPPYTRKVVKRFVGMRTLRNVPMLSQVDGRVLKMMCDYLKPVKYQENTMVFETGVPFDRMVFITDGSMWTYTIATDHQTSHSGKATSGGLVTSSSTEAHFLKKGDAYGHMLLQLASSSSTAVPISVANVRCHTKVEAFVLMANDLRNIVTACGDLWPAFDYKASQDQAADQMAPPGHFQQQQGPKKRTVASSNIHPTGYSG

SEQ ID NO: 5 - DNA, 1947 bp, Fragaria sp.
  source 1..1947: mol_type = other DNA
atggctaaacatcagggaccaaccaatgaaatccaaatgagcattacaccagatgtgtcttatgatttccatagtcctgaggctctaaaaccgacacccgaacatggacaacctcaagcaagcagcaaaagacggaattggccaacagttgccggagtttcttcagctttgaacccatggtggaataagatactggtaacttcatgtgtgattgcagttttatttgatcccttgttcttttacattccatacaccagcgaggaaaaaaagtgtatgggaaacgacgaaagagtgcagactccagctctgattttccgatcagtcacagacatcattttcctggtgcatatcatataccaactatatgtggctatcaaatttgcactctcaagggtttccgaggatgaaagtttggtttttagatatgaatcggtatcccgagagcaaatgattcagtttgcgaaggcattttctcacaagttgtcatggcgatctttcctaactgacgttttcgctattttgcccataccacaactgctaatagtaagattgtttttcaacattggaggcaataactacttgttaaaaagaaagattgtgagcggttttcttctaatccaatatgtgccaagaatctatcgaatttatctttcatctgtcgcggtcacagaatctgcaatgtgggttagaggtgtatttaacatttttctctacattcttgcgagtcatgcacttggagccttttggtacttttgttctattcagcgagagacattatgttggtatcgaacttgcctcgctacaaatatatcagaatcaaaatgcaccttttactgccataacagtgctgctgcacttatgaccccacaattcaagacaactctagacgagaaatgcattctaaaagttccatataacatgaccgatccaccatatgattttggaatattttttgatgccctcaaaaatgatatccaagggaaaataaatgttccacaaaagattggttactgtttttggtggggcttgcgaaacttgagtaattttggtacaaatttggaaacaagtacctatctgtgggaaaacagctttgccattttaatttctatcatcggcttgctgttgtttttatacctcattggaaatgtacagatttatatgcagagggcagttaccagagcggaggaggtaagagagaagcttcggatcaaaaagaacgatatagataattggatccaaaataatggtatcgagaatgacatgaaggaagagatcatgaaaaacatcgttgaaaagttggaaaacgacataagtgcagatctggatgatatatactctattcttcccccgtataccaggaaagttgtgaagcgatttgtcggcatgaggacgctaagaaatgtaccaatgcttagccaggtggatggaagagttttgaagatgatgtgcgactatctgaagccggtcaagtaccaggagaatacaatggtttttgaaacgggagtaccatttgatagaatggtcttcattacagatgggtcaatgtggacttacacgattgctactgatcatcagactagtcattctggaaaagcaacgtcaggaggactagttacttcttcgtccacggaagctcacttcctcaagaagggtgatgcttatggacacatgcttctgcaattagcatcatcatcttctacggcggttcctatctcagttgcaaatgtcaggtgccacacaaaggtagaggcgtttgtcctcatggccaatgacttgagaaacatagtaactgcgtgcggagatttgtggccggcttttgactacaaggcttctcaagatcaggcggctgatcagatggcaccgccggggcattttcagcaacaacaagggccaaagaagcgtactgtagcatcatcaaatatccatcctaccggctacagtgggtga

SEQ ID NO: 6 - protein, 648 aa, Fragaria sp.
MAKHQGPTNEIQMSITPDVSYDFHSPEALKPTPEHGQPQASSKRRNWPTVAGVSSALNPWWNKILVTSCVIAVLFDPLFFYIPYTSEEKKCMGNDERVQTPALIFRSVTDIIFLVHIIYQLYVAIKFALSRVSEDESLVFRYESVSREQMIQFAKAFSHKLSWRSFLTDVFAILPIPQLLIVRLFFNIGGNNYLLKRKIVSGFLLIQYVPRIYRIYLSSVAVTESAMWVRGVFNIFLYILASHALGAFWYFCSIQRETLCWYRTCLATNISESKCTFYCHNSAAALMTPQFKTTLDEKCILKVPYNMTDPPYDFGIFFDALKNDIQGKINVPQKIGYCFWWGLRNLSNFGTNLETSTYLWENSFAILISIIGLLLFLYLIGNVQIYMQRAVTRAEEVREKLRIKKNDIDNWIQNNGIENDMKEEIMKNIVEKLENDISADLDDIYSILPPYTRKVVKRFVGMRTLRNVPMLSQVDGRVLKMMCDYLKPVKYQENTMVFETGVPFDRMVFITDGSMWTYTIATDHQTSHSGKATSGGLVTSSSTEAHFLKKGDAYGHMLLQLASSSSTAVPISVANVRCHTKVEAFVLMANDLRNIVTACGDLWPAFDYKASQDQAADQMAPPGHFQQQQGPKKRTVASSNIHPTGYSG

SEQ ID NO: 7 - protein, 798 aa, Fragaria sp.
MALHGRMLLLQLSLAAVLLATTMLAAAQAPPPDCNESCGGVSVPYPFGLTEGCYLHVPGQDSQQPFKITCNTTTSQPSLQFSDSDNFPTNIANIFVEESALQVMMAPSYNCYTNNTYDSSMDKVMDLNLPPSFTLSDRNKVYNLGCNKVTKLIGYPLEVTDPDQLFEVGFSGVSLCQDEFGKRFPDTCTGFGCSVNSIPIGLQNITLGVWTLGTSLGTTAQWGLSYPCSYGFVVDERNFTFAGNQSFDALRSGEIKKLPVLANWAIGNDNCEAAKKKNETAFACKTVNSKCVDRAGGGYFCQCEDGYEGNPYLLDNCLDINECNKNSTLCSGPATCINSIGGYTCKCHKGYRNDDHDKNKCVQITETSSKNDKEMKISLGVSLSFLVILIITFSIYCVMKRRKFKQLCDKYYKDNGGFLLPQEMQKYRGSQAPRIFKLEELNKATNKFDVTNIIGEGGFGLVYKGTLPDNKQVVAIKKSKTDAPTMTPENRIAHTKQFINEMIVLSGIKHRNVVRLLGCCLETKTPILVYEFVSNGTLYEHIHKKKGQGPLSFEQRVKIAAETAGSLAYLHYSASSTPILHRDVKASNILLDENCIAKVSDFGASKLVPEDENTQMATLVQGTLGYLDPEYLQSHALTEKSDVYSFGVVLVELITSQVAISNKKLEAEKNLANCFLKSVADNNLDQILDHEIIKEESFEIAEQVAHLAKRCLSLKGEDRPTMEEVETELKVILAVMAKHPGGKPDSSPKETDYLLAASPSNAYVVDVRSDEGEVITSIDYDKSMQNQAQMMKHYDGGR

(19) United States
(12) Patent Application Publication
(10) Pub. No.: US 2026/0146258 A1
(43) Pub. Date: May 28, 2026

(54) COMPOSITIONS AND METHODS FOR INCREASING PHYTOPHTHORA CROWN ROT RESISTANCE

(71) Applicant: University of Florida Research Foundation, Incorporated, Gainesville, FL (US)

(72) Inventors: Seonghee Lee, Lithia, FL (US); Vance M. Whitaker, Brandon, FL (US); Hyeondae Han, Riverview, FL (US); Yoon Jeong Jang, Riverview, FL (US); Youngjae Oh, Cheongju (KR)

(21) Appl. No.: 19/119,495

(22) PCT Filed: Oct. 27, 2023
(86) PCT No.: PCT/US2023/078005

§ 371 Date: Apr. 9, 2025

Related U.S. Application Data
(60) Provisional application No. 63/381,447, filed on Oct. 28, 2022.

(51) Int. Cl. C12N 15/82; C07K 14/415; C12N 9/12; C12N 9/22; C12N 15/11

(52) U.S. Cl. CPC C12N 15/8282; C07K 14/415; C12N 9/12; C12N 9/226; C12N 15/111; C12N 15/8213; C12N 2310/20

(57) ABSTRACT

Described are CRISPR constructs and systems that can be used to generate Phytophthora crown rot resistant strawberry cultivars rapidly and efficiently.

### CROSS-REFERENCE TO RELATED APPLICATIONS

This application claims the benefit of U.S. Provisional Application No. 63/381,447, filed Oct. 28, 2022, and is incorporated herein by reference.

### STATEMENT REGARDING FEDERALLY SPONSORED RESEARCH OR DEVELOPMENT

This invention was made with government support under Grant No. 2017-51181-26833 and No. 2022-51181-38328 awarded by The United States Department of Agriculture, National Institutes of Food & Agriculture. The government has certain rights in the invention.

This invention was made in whole or in part from funding received under a contract from the Florida Strawberry Research & Education Foundation.

### SEQUENCE LISTING

The Sequence Listing written in file “T18872WO001_SeqListing_ST26.xml is 16 kilobytes, was created on Sep. 28, 2023, and is hereby incorporated by reference.

### BACKGROUND

Strawberry phytophthora crown rot (PhCR) disease is caused by Phytophthora cactorum, the oomycete fungal pathogen and hemi-biotrophic found in nature. It was first reported as a causative agent for strawberry crown rot disease in Germany in 1952 and is considered one of the most significant soil-bome diseases affecting the strawberry in temperate parts of the world, causing up to 40% loss in strawberry production. Strawberry crown rot disease causes stunted growth, wilting of leaves, and ultimately leads to the collapse and death of the plant. Reddish brown wounds and longitudinal splits are also visible within the crown. P. cactorum can remain alive in soil for long periods of time and poses a threat for proliferation of plants due to rapid dissemination of infection. Fumigation of soil with methyl bromide, dazomet, and chloropicrin 1,3-dichloropropene was the most frequently used method for controlling P. cactorum. However, the discontinuation of these fumigants has worsened the situation for controlling P. cactorum. Thus, there is an increased the need for deploying new disease resistant cultivars to manage crown and root rot disease.

The first whole genome and SNP genotyping platform, ‘Axiom® IStraw90 array’ and ‘IStraw35 SNP array,’ used high-quality SNP markers that precisely located to the octoploid genetic linkage map derived from F. vesca genome. This platform has been used for high-density scanning of large populations and has identified many major quantitative trait loci (QTL) for fruit quality and disease resistance. Since the most of SNP probes from Axiom® IStraw90 and IStraw35 arrays were developed from the diploid strawberry F. vesca genome, it was difficult to find the correct sub-genome in allo-octoploid strawberry due to differences in ploidy levels inherited from the other four ancestors.

Recently, a FanaSNP array, with 50,000 subgenome-specific SNPs, was developed from the octoploid strawberry reference genome ‘Camarosa.’ The availability of this new reference genome and SNP array have accelerated the identification of genes and loci controlling important traits for disease resistance and fruit quality in the cultivated strawberry. QTL associated with PhCR resistance have been recently identified and introgressed for PhCR resistance breeding in cultivated octoploid strawberry (F. x ananassa). In the diploidy strawberry, F. vesca, Davik and colleagues reported the resistance locus of Phytophthora cactorum 1 (RPc-1) (Davik et al., 2015). In the cultivated octoploid strawberry, Mangandi and colleagues reported major resistance locus, FaRPc2, located in a linkage group (LG) 7D confers resistance against P. cactorum using a pedigree-based analysis in complex, multiparental population sets (Mangandi et al., 2017). The two predominant SNP haplotypes in the major locus FaRPc2, haplotypes FaRPc2-H2 and FaRPc2-H3, are tightly connected with varied intensity of resistance against P. cactorum. In addition, three major loci, FaRPc6C, FaRPc6D, and FaRPc7D, identified using bi-parental population (‘Emily’ x ‘Fenella’), separate for resistance to P. cactorum in European germplasm.

Genetic and molecular studies to increase resistance to P. cactorum have focused on finding resistance genes or resistance loci that can be introgressed into cultivars using molecular marker techniques. Combining the resistance haplotypes of H2 and H3 has been reported to provide more effective and durable resistance, although the H3 resistance effect is larger than that of H2. The two resistant haplotypes of FaRPc2 may be derived from two different sources of resistance, and it is possible that two distinct functional resistance alleles may exist at FaRPc2. In the University of Florida (UF) breeding program, haplotypes FaRPc2-H2 and FaRPc2-H3 were found to occur mainly in different pedigree lineages, and these two haplotypes are almost always inherited through separate lineages. The FaRPc2-H3 genotype is the major resistance haplotype in cultivated strawberry in the U.S., while FaRPc2-H2 is more frequently present in European accessions. The University of California, Davis (UCD) cultivar ‘Camarosa,’ possesses the resistant FaRPc2-H3 genotype. Some of the early UF strawberry breeding programs have been initiated using ‘Camarosa,’ therefore, FaRPc2-H3 from ‘Camarosa’ is a major source of PhCR resistance in U.S. breeding programs.

Genetic variation underlying the difference in resistance associated with this haplotype is not well understood. The functional underpinnings of H2 resistance compared to h2 susceptibility and H3 resistance remain uncharacterized. Questions surrounding these resistance and susceptibility haplotypes will likely not be solved until the resistance gene or genes in the FaRPc2 region are cloned. Moreover, ‘Camarosa’ is an H3-homozygote resistant cultivar, which makes it insufficient at the chromosomal level to study H2 resistance. To overcome these problems, we have assembled a chromosome-scale and haplotype-phased genome assembly using the accession FL 16.33-8, which possess the different haplotypes for FaRPc2-H2 and H3.

The cultivated strawberry (2n=8 x=56) has a complex allo-octoploid genome with four sets of identical homologous diploid chromosomes and a total genome size of ˜813.4 Mb. Research in the octoploid strawberry has long been hampered by the unavailability of a high-quality reference genome. The recent availability of the chromosome-scale F. x ananassa ‘Camarosa’ reference genome and another octoploid reference genome from the Japanese cultivar ‘Reikou’ have finally allowed the field to advance. The most recent chromosome level assembly of an octoploid strawberry genome was performed in a highly homozygous inbred line, ‘Wongyo 3115,’ using long- and short-read sequencing. However, the reference genomes of ‘Camarosa’ and ‘Wongyo 3115’ are unphased genomes, in that they have merged homologous chromosomes into one sequence. By contrast, the reference genome for ‘Reikou’ is haplotype phased, but this draft genome sequence has not been fully characterized. In an unphased genome, a consensus sequence is obtained from the two or more genomes comprising the hybrids/polyploids by collapsing the homologous/homoeologous sequences. In a phased genome, all haplotype/subgenome sequences of the multiple genomes in hybrids/polyploids are individually determined. Haplotype phasing is, therefore, an essential component of phased sequencing analysis.

A recent haplotype phased genome of a cultivar from UCD, Royal Royce, used the trio binning technique (Koren et al. 2018), which limited confounding effects from heterozygosity and avoided co-assembly of alleles. In the allo-octoploid strawberry with a high ploidy level and highly heterozygous genome, trio binning assembly using long-read sequencing technologies such as Pacific Biosciences (PacBio), combined with short read Illumina sequence data of each parent genome, is the most comprehensive method to provide unbiased DNA sequences on the chromosome scale. This haplotype-phased genome is the key for the advancement of genomic studies of the allo-octoploid strawberry, and in this regard, the new reference genome of the Royal Royce cultivar established the nomenclature for each sub-genome in an octoploid strawberry.

In the FaRPc2 resistance region against P. cactorum in the octoploid strawberry, there are no reports of transcriptome analysis to study defense related genes. Prior transcriptome studies of P. cactorum infection were carried out in diploid strawberry, but regulation of defense response during infection might differ from diploid F. vesca in allo-octoploid strawberry due to differences in polyploidy and development characteristics. Thus, the availability of an octoploid transcriptome reference sequence aids in mapping the transcriptomic data of F. x ananassa to a reference to decipher genes critical for defense against P. cactorum. There remains an unmet need for better understanding of the genes responsible for resistance against PhCR in order to generate resistant crops and preserve strawberry production.

### SUMMARY

Strawberry phytophthora crown rot disease, caused by P. cactorum, is considered one of the most significant soil-borne diseases affecting the strawberry (Fragaria). Described are compositions and methods for generating genetically modified PhCR resistant Fragaria plants. Also described are markers for use in selecting PhCR resistant Fragaria plants. The modified plants can be used to introgress PhCR resistance into other genetic backgrounds.

Described are a locus, haplotypes, and specific genes responsible for increasing PhCR resistance in octoploid Fragaria. The Fragaria Resistance to Phytophthora cactorum locus/gene 2 (FaRPc2) locus is located on chromosome 7-3 of the allo-octoploid strawberry genome. This FaRPc2 region comprises four predominant SNP haplotypes, namely, H1, H2, H3, and H4. Two haplotypes, H2 and H3, are strongly linked with PhCR resistance. The H3 haplotype exhibits a larger resistance effect than H2, Within the H3 haplotype, three genes play a significant role in mediating PhCR resistance: WAK1, CNGC1, and CNGC2. In some embodiments, one or more of a heterologous WAK1 gene, a heterologous CNGC1 gene, and a heterologous CNGC2 gene may be introduced into a Fragaria plant to confer PhCR resistance. In some embodiments, one or more of a heterologous WAK1 gene, a heterologous CNGC1 gene, and a heterologous CNGC2 gene may be introduced into an octoploid Fragaria plant to confer PhCR resistance. In some embodiments, one or more genetic markers associated with each of the WAK1 gene, the CNGC1 gene, and/or the CNGC2 gene are used to select for cultivars or progeny plants resistant to PhCR. In some embodiments, one or more genetic markers associated with each of the WAK1 gene, the CNGC1 gene, and/or the CNGC2 gene are used to select for cultivars or progeny octoploid Fragaria plants resistant to PhCR. In some embodiments, the WAK1 gene comprises SEQ ID NO: 1. In some embodiments, the CNGC1 gene comprises SEQ ID NO: 3. In some embodiments, the CNGC2 gene comprises SEQ ID NO: 5.

In some embodiments, a heterologous WAK1 gene is introduced into a Fragaria plant to confer PhCR resistance. In some embodiments, a heterologous WAK1 gene is introduced into an octoploid Fragaria plant to confer PhCR resistance. In some embodiments, a heterologous CNGC1 gene is introduced into a Fragaria plant to confer PhCR resistance. In some embodiments, a heterologous CNGC1 gene is introduced into an octoploid Fragaria plant to confer PhCR resistance. In some embodiments, a heterologous CNGC2 gene is introduced into a Fragaria plant to confer PhCR resistance. In some embodiments, a heterologous CNGC2 gene is introduced into an octoploid Fragaria plant to confer PhCR resistance.

In some embodiments, two or more of a heterologous WAK1 gene, a heterologous CNGC1 gene, and a heterologous CNGC2 gene may be introduced into a Fragaria plant to confer PhCR resistance. In some embodiments, one or more of a heterologous WAK1 gene, a heterologous CNGC1 gene, and a heterologous CNGC2 gene may be introduced into an octoploid Fragaria plant to confer PhCR resistance. In some embodiments, a heterologous WAK1 gene and a heterologous CNGC1 gene are introduced into a Fragaria plant to confer PhCR resistance. In some embodiments, a heterologous WAK1 gene and a heterologous CNGC1 gene are introduced into an octoploid Fragaria plant to confer PhCR resistance. In some embodiments, a heterologous WAK1 gene and a heterologous CNGC2 gene are introduced into a Fragaria plant to confer PhCR resistance. In some embodiments, a heterologous WAK1 gene and a heterologous CNGC2 gene are introduced into an octoploid Fragaria plant to confer PhCR resistance. In some embodiments, a heterologous CNGC1 gene and a heterologous CNGC2 gene are introduced into a Fragaria plant to confer PhCR resistance. In some embodiments, a heterologous CNGC1 gene and a heterologous CNGC2 gene are introduced into an octoploid Fragaria plant to confer PhCR resistance.

In some embodiments, a heterologous WAK1 gene, a heterologous CNGC1 gene, and a heterologous CNGC2 gene are introduced into a Fragaria plant to confer PhCR resistance. In some embodiments, a heterologous WAK1 gene, a heterologous CNGC1 gene, and a heterologous CNGC2 gene are introduced into an octoploid Fragaria plant to confer PhCR resistance.

In some embodiments, one or more genetic markers associated with the WAK1 gene are used to select for cultivars or progeny plants resistant to PhCR. In some embodiments, one or more genetic markers associated with the WAK1 gene are used to select for cultivars or progeny octoploid Fragaria plants resistant to PhCR. In some embodiments, one or more genetic markers associated with the CNGC1 gene are used to select for cultivars or progeny plants resistant to PhCR. In some embodiments, one or more genetic markers associated with the CNGC1 gene are used to select for cultivars or progeny octoploid Fragaria plants resistant to PhCR. In some embodiments, one or more genetic markers associated with the CNGC2 gene are used to select for cultivars or progeny plants resistant to PhCR. In some embodiments, one or more genetic markers associated with the CNGC2 gene are used to select for cultivars or progeny octoploid Fragaria plants resistant to PhCR.

In some embodiments, one or more genetic markers associated with each of the WAK1 gene and the CNGC1 gene are used to select for cultivars or progeny plants resistant to PhCR. In some embodiments, one or more genetic markers associated with each of the WAK1 gene and the CNGC1 gene are used to select for cultivars or progeny octoploid Fragaria plants resistant to PhCR. In some embodiments, one or more genetic markers associated with each of the WAK1 gene and the CNGC2 gene are used to select for cultivars or progeny plants resistant to PhCR. In some embodiments, one or more genetic markers associated with each of the WAK1 gene and the CNGC2 gene are used to select for cultivars or progeny octoploid Fragaria plants resistant to PhCR. In some embodiments, one or more genetic markers associated with each of the CNGC1 gene and the CNGC2 gene are used to select for cultivars or progeny plants resistant to PhCR. In some embodiments, one or more genetic markers associated with each of the CNGC1 gene and the CNGC2 gene are used to select for cultivars or progeny octoploid Fragaria plants resistant to PhCR.

In some embodiments, one or more genetic markers associated with each of the WAK1 gene, the CNGC1 gene, and the CNGC2 gene are used to select for cultivars or progeny plants resistant to PhCR. In some embodiments, one or more genetic markers associated with each of the WAK1 gene, the CNGC1 gene, and the CNGC2 gene are used to select for cultivars or progeny octoploid Fragaria plants resistant to PhCR.

Described are methods of targeted DNA insertion in plants that can be used to generate PhCR resistant plants more rapidly and efficiently than standard breeding programs through favorable trait and/or marker-based breeding schemes. These DNA insertion methods can be used to target the FaRPc2 locus in Fragaria plants and improve resistance against P. cactorum infestation. In some embodiments, targeting the FaRPc2 locus involves inserting one or more of a WAK1 gene, a CNGC1 gene, and a CNGC2 gene, resulting in improved resistance against P. cactorum infestation and PhCR.

In some embodiments, targeting the FaRPc2 locus involves inserting a WAK1 gene, resulting in improved resistance against P. cactorum infestation and PhCR. In some embodiments, targeting the FaRPc2 locus involves inserting a CNGC1 gene, resulting in improved resistance against P. cactorum infestation and PhCR. In some embodiments, targeting the FaRPc2 locus involves inserting a CNGC2 gene, resulting in improved resistance against P. cactorum infestation and PhCR. In some embodiments, targeting the FaRPc2 locus involves inserting two or more of a WAK1 gene, a CNGC1 gene, and a CNGC2 gene, resulting in improved resistance against P. cactorum infestation and PhCR. In some embodiments, targeting the FaRPc2 locus involves inserting a WAK1 gene and a CNGC1 gene, resulting in improved resistance against P. cactorum infestation and PhCR. In some embodiments, targeting the FaRPc2 locus involves inserting a WAK1 gene and a CNGC2 gene, resulting in improved resistance against P. cactorum infestation and PhCR. In some embodiments, targeting the FaRPc2 locus involves inserting a CNGC1 gene and a CNGC2 gene, resulting in improved resistance against P. cactorum infestation and PhCR. In some embodiments, targeting the FaRPc2 locus involves inserting a WAK1 gene, a CNGC1 gene, and a CNGC2 gene, resulting in improved resistance against P. cactorum infestation and PhCR.

In some embodiments, strawberry plants expressing one or more of: a heterologous WAK1 gene, a heterologous CNGC1 gene, and a heterologous CNGC2 gene are described. The strawberry plants can be octoploid strawberry plants, including allo-octoploid strawberry plants. In some embodiments, strawberry plants expressing a heterologous WAK1 gene are described. In some embodiments, strawberry plants expressing a heterologous CNGC1 gene are described. In some embodiments, strawberry plants expressing a heterologous CNGC2 gene are described. The strawberry plants can be octoploid strawberry plants, including allo-octoploid strawberry plants.

In some embodiments, strawberry plants expressing two or more of: a heterologous WAK1 gene, a heterologous CNGC1 gene, and a heterologous CNGC2 gene are described. In some embodiments, strawberry plants expressing a heterologous WAK1 gene and a heterologous CNGC1 gene are described. In some embodiments, strawberry plants expressing a heterologous WAK1 gene and a heterologous CNGC2 gene are described. In some embodiments, strawberry plants expressing a heterologous CNGC1 gene and a heterologous CNGC2 gene are described. The strawberry plants can be octoploid strawberry plants, including allo-octoploid strawberry plants.

In some embodiments, strawberry plants expressing a heterologous WAK1 gene, a heterologous CNGC1 gene, and a heterologous CNGC2 gene are described. The strawberry plants can be octoploid strawberry plants, including allo-octoploid strawberry plants.

Described are methods of increasing resistance of strawberry plants to phytophthora crown rot (PhCR) disease comprising expressing in the strawberry plants one or more heterologous genes selected from the group consisting of: a WAK1 gene, a CNGC1 gene, and a CNGC2 gene. In some embodiments, the methods comprise expressing a heterologous WAK1 gene in the strawberry plants. In some embodiments, the methods comprise expressing a heterologous CNGC1 gene in the strawberry plants. In some embodiments, the methods comprise expressing a heterologous CNGC2 gene in the strawberry plants. In some embodiments, the methods comprise expressing two or more of a heterologous WAK1 gene, a heterologous CNGC1 gene, and a heterologous CNGC2 gene in the strawberry plants. In some embodiments, the methods comprise expressing a heterologous WAK1 gene and a heterologous CNGC1 gene in the strawberry plants. In some embodiments, the methods comprise expressing a heterologous WAK1 gene and a heterologous CNGC2 gene in the strawberry plants. In some embodiments, the methods comprise expressing a heterologous CNGC1 gene and a heterologous CNGC2 gene in the strawberry plants. In some embodiments, the methods comprise expressing a heterologous WAK1 gene, a heterologous CNGC1 gene, and a heterologous CNGC2 gene in the strawberry plants.

The methods can be used to improve strawberry crop yield in areas where P. cactorum is present. In some embodiments, loss of crop yield in genetically modified PhCR resistant plants may be reduced by at least 5%, at least 10%, at least 15%, at least 20%, at least 25%, at least 30%, at least 40%, at least 50%, at least 60%, at least 70%, at least 80%, at least 90%, or at least 95%, or up to 100% compared to P. cactorum susceptible Fragaria cultivars. The methods can be used to decrease the incidence or severity of phytophthora crown rot in areas where P. cactorum is present by at least 10%, at least 5%, at least 10%, at least 15%, at least 20%, at least 25%, at least 30%, at least 40%, at least 50%, at least 60%, at least 70%, at least 80%, at least 90%, or at least 95%, or up to 100% compared to P. cactorum susceptible Fragaria cultivars. In some embodiments, the methods can be used to reduce loss of crop yield due to PhCR in strawberry plants by at least 5%, at least 10%, at least 15%, at least 20%, at least 25%, at least 30%, at least 40%, at least 50%, at least 60%, at least 70%, at least 80%, at least 90%, or at least 95%, or up to 100% relative of similar strawberry plants that do not express the heterologous WAK1 gene, the heterologous CNGC1 gene, and/or the heterologous CNGC2 gene. In some embodiments, the methods can be used to decrease the incidence or severity of phytophthora crown rot in strawberry plants by at least 10%, at least 5%, at least 10%, at least 15%, at least 20%, at least 25%, at least 30%, at least 40%, at least 50%, at least 60%, at least 70%, at least 80%, at least 90%, or at least 95%, or up to 100% relative of similar strawberry plants that do not express the heterologous WAK1 gene, the heterologous CNGC1 gene, and/or the heterologous CNGC2 gene.

CRISPR constructs and systems comprising Cas proteins and guide RNAs for directed modification at the FaRPc2 locus to confer PhCR resistance are described. The CRISPR constructs and systems can be used for directed modifications at the FaRPc2 locus that include insertions, deletions, or mutations. In some embodiments, the CRISPR constructs and systems insert one or more of a heterologous WAK1 gene, a heterologous CNGC1 gene, and a heterologous CNGC2 gene. The heterologous gene can be inserted at the FaRPc2 or at another location in the plant genome, such as a safe haven site. In some embodiments, CRISPR constructs and systems are described that can be used to knock in one or more of a heterologous WAK1 gene, a heterologous CNGC1 gene, and a heterologous CNGC2 gene into a genome of a plant (e.g., a strawberry plant). The heterologous WAK1 gene, the heterologous CNGC1 gene, and/or the heterologous CNGC2 gene can be inserted into the FaRPc2 locus or any site in the plant genome suitable of insertion and expression of a heterologous gene (i.e., a safe haven site).

In some embodiments, the CRISPR constructs and systems are used to generate genetically modified Fragaria plant cells carrying one or more of a heterologous WAK1 gene, a heterologous CNGC1 gene, and a heterologous CNGC2 gene (i.e., PhCR resistance-associated genes). These plant cells can then be used to produce regenerant progeny transgenic plants that are PhCR resistant. Any of the described CRISPR constructs and systems, as well as any of the other methods of targeted DNA insertion in plants, can be used to generate the transgenic Fragaria plant resistant to PhCR.

A Fragaria plant can be any member of the Fragaria genus suitable for commercial strawberry production. A Fragaria plant can be a diploid species, a tetraploid species, a hexaploid species, an octoploid species, or any other ploidy species or hybrid thereof. A Fragaria plant can be, but is not limited to, Fragaria vesca, Fragaria x bifera. Fragaria x bringhurstii, Fragaria virginiana, Fragaria chiloensis, or Fragaria x ananassa. In some embodiments, the Fragaria plant is a Fragaria x ananassa plant.

### BRIEF DESCRIPTION OF THE FIGURES

FIG. 1A. Illustration showing fine mapping of the FaRPc2 region responsible for conferring resistance to PhCR. Diagram showing fine mapping of the H3 region of FaRPc2. Preliminary mapping of FaRPc2 region using 11 markers derived from Axiom® IStraw90 SNP probes. The FaRPc2 region of H3 is reduced by new subgenome specific HRM markers derived from the 50k FanaSNP array. A=AX-89845076, B=AX-89845054, C=AX-89801722, D=AX-184233889, E=AX-184474004, F=AX-184173618, G=AX-184664523, H=AX-89844925. I=AX-89801602. J=AX-89902166. K=AX-89902115, L=AX-89844827, M=AX-89844806, N=AX-89902057, and O=AX-89844809. Bold font indicates new subgenome specific HRM markers.

FIG. 1B. Illustration showing fine mapping of the FaRPc2 region responsible for conferring resistance to PhCR. Table showing association of HRM marker alleles with SNP haplotype in the FaRPc2-H3 region. Notes: a, Based on the area under the disease progress curve (AUDPC) values reported in Mangandi et al. (2017) b, Diplotypes of octoploid strawberry accessions in the FaRPc2 region from Mangandi et al. (2017) and their overall associations with resistance (R), high susceptibility (HS), and susceptibility (S) phenotypes.

FIG. 2A. Diagrams showing the haplotype-phased genome of FL 16.33-8. Diagram showing genome visualization of haplotype-phased genome A of FL 16.33-8. The tracks from outside to inside are: a=haplotype-phased subgenome name of FL 16.33-8, b=length (Mb) of sub-genomes, c=number of genes per 1 Mb window, d=density of long term repeated (LTR) per 1 Mb window, e=LTR assembly index (LAI) in sub-genomes 1Ab to 7Db plotted in 1 Mb window, f=position of the indel that differed by more than 30,000 bp among ‘Royal Royce’, ‘Wongyo 3113’, and FL 16.33-8, g=synteny comparison within the haplotype-phased genome

FIG. 2B-C. Diagrams showing the haplotype-phased genome of FL 16.33-8. (B) K-mer blob plot showing for each scaffold the maternal (16.33-8.asm.hap1.p_ctg; circles along X axis) and paternal (16.33-8.asm.hap2.p_ctg; circles along y axis) assembly and how many k-mers in that scaffold are found in the matemal or patemal k-mer sets. The size of the blob represents the total number of k-mers in that scaffold. There is good separation between the haplotypes, with each assembly consisting mostly of k-mers associated with the associated parental k-mer. (C) Graph illustration a combined spectra plot of inherited K-mers. The first peak corresponds to k-mers present in the raw reads but missing from the assembly due to sequencing errors, the second peak corresponds to k-mers from heterozygous regions, and the third peak corresponds to k-mers from homozygous regions. These plots show a complete and well-separated assembly of both haplotypes in the F1 offspring genome.

FIG. 3. Graphs illustrating visualization of comparative genomic analysis. A. Graphs illustrating comparison of the collinearity between the genetic linkage map and haplotype-phased genome A of FL 16.33-8. Genetic positions from a genetic linkage map for ‘Brilliance’ x FL 16.33-8 plotted against the corresponding physical positions in the ‘Florida Brilliance’ genome assembly (blue points). X-axis indicate cM of genetic linkage map, Y-axis indicate genome coordinates in the haplotype-phased genome A of FL 16.33-8. B. Graphs illustrating dot plot visualization of whole-genome alignment of the FL 16.33-8 Haplotype phased genome A assembly to the diploid F. vesca genome assembly (v4.0) using MCScanX.

FIG. 4. Illustration showing characterization of genomic architecture of the FaRPc2 region. A Mauve alignment of genomic sequence of FaRPc2_H3 region among ‘Florida Brilliance’, FL 16.33-8 hap A. Royal Royce hap A and B. and ‘Camarosa’. The shaded blocks represent homology among the genomic sequences of FaRPc2_H3 region by Mauve alignment on default settings. Inside each block, Mauve draws a similarity profile of the genome sequence. Lines indicate which regions in each genomic sequence are homologous. White boxes indicate sequences different among genotypes.

FIG. 5A. Graphs showing RNA-Seq expression analysis between FaRPc2-H1 and FaRPc2-H3. Heatmap of differentially expressed genes at the whole-genome level with PI treatment.

FIG. 5B-C. Graphs showing RNA-Seq expression analysis between FaRPc2-H1 and FaRPc2-H3. (B) Venn diagram illustrating analysis of upregulated DEGs in the water and PI treatments between FaRPc2-H1 and FaRPc2-H3 genotypes. (C) Volcano plot comparing gene expression levels between FaRPc2-H1 PI and FaRPc2-H3 PI treatments.

FIG. 5D. Graphs showing RNA-Seq expression analysis between FaRPc2-H1 and FaRPc2-H3. Heatmap illustrating visualization of the genes present in 546 kb of FaRPc2-H3 region.

FIG. 6A. Sequence alignment of candidate genes, WAK, CNGC1, and CNGC2, in a major locus FaRPc2-H3 responsible for resistance against P. cactorum. Graphical representation of the location of candidate genes, WAK, CNGC1, and CNGC2, in a major locus FaRPc2-H3.

FIG. 6B. Amino acid sequence alignment of WAK, in a major locus FaRPc2-H3 responsible for resistance against P. cactorum. Residues that are conserved across all sequences are highlighted in black. Below the protein sequences is a key denoting conserved sequence (|), conservative mutations (:), semi-conservative mutations (.), and non-conservative mutations ( ). Brilliance_WAK=SEQ ID NO: 7: FL 16.33-8_WAK=SEQ ID NO: 2.

FIG. 6C. Amino acid sequence alignment of CNGC1 and CNGC2, in a major locus FaRPc2-H3 responsible for resistance against P. cactorum. Residues that are conserved across all sequences are highlighted in black. Below the protein sequences is a key denoting conserved sequence (|), conservative mutations (:), semi-conservative mutations (.), and non-conservative mutations (. FL 16.33-8_CNGC1=SEQ ID NO: 4; FL 16.33-8_CNGC2=SEQ ID NO: 6.

FIG. 7A. Illustrations showing Agrobacterium-mediated transient RNAi gene silencing of WAK and CNGC in in F. x ananassa. Diagram illustrating FaCNGC and FaWAK construction of pK7GWIWG(II) RNAi vectors for transient gene silencing in F. x ananassa.

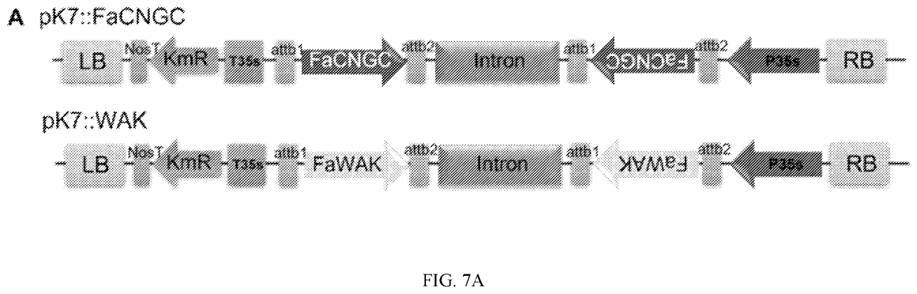

FIG. 7B-C. Graphs illustrating Agrobacterium-mediated transient RNAi gene silencing of WAK and CNGC in in F. x ananassa. (B) Graphs illustrating the result of the silenced transient assay in strawberries used for relative expression vector validation between empty vector (EV) and FaWAK and FaCNGC. (C) Graphs illustrating the disease severity incidence rate of Phytophthora cactorum. The bars represent the standard error of three biological replicates with three technical replicates. The asterisks mark indicated a significant difference from the EM (***P<0.001, student's T-test).

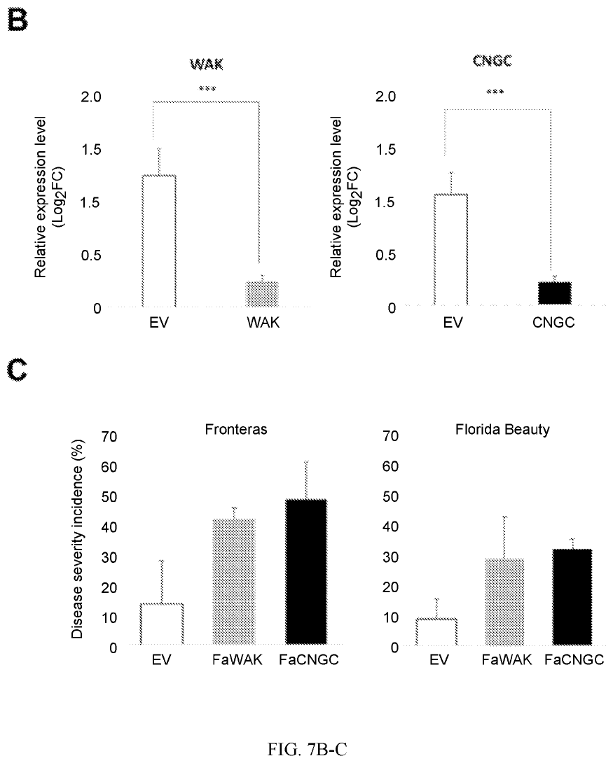

FIG. 7D. Images showing Agrobacterium-mediated transient RNAi gene silencing of WAK and CNGC in in F. x ananassa. Images illustrating representative phenotypes of strawberry root and crown inoculated with Phytophthora cactorum.

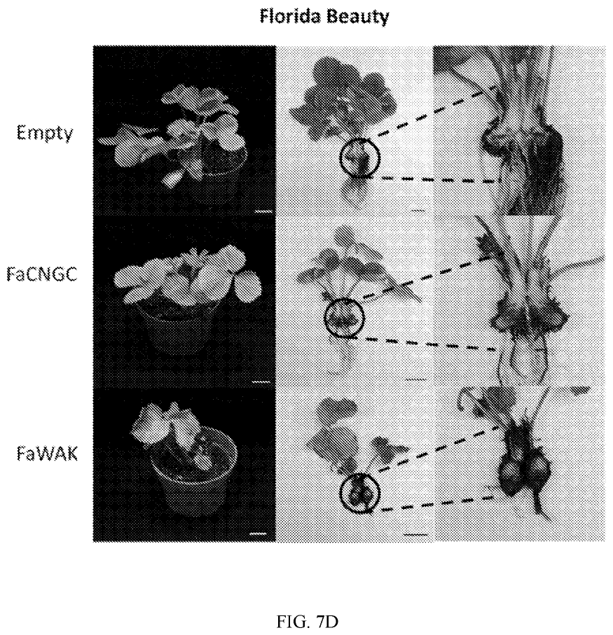

FIG. 7E. Images showing Agrobacterium-mediated transient RNAi gene silencing of WAK and CNGC in in F. x ananassa. Images illustrating representative phenotypes of strawberry root and crown inoculated with Phytophthora cactorum.

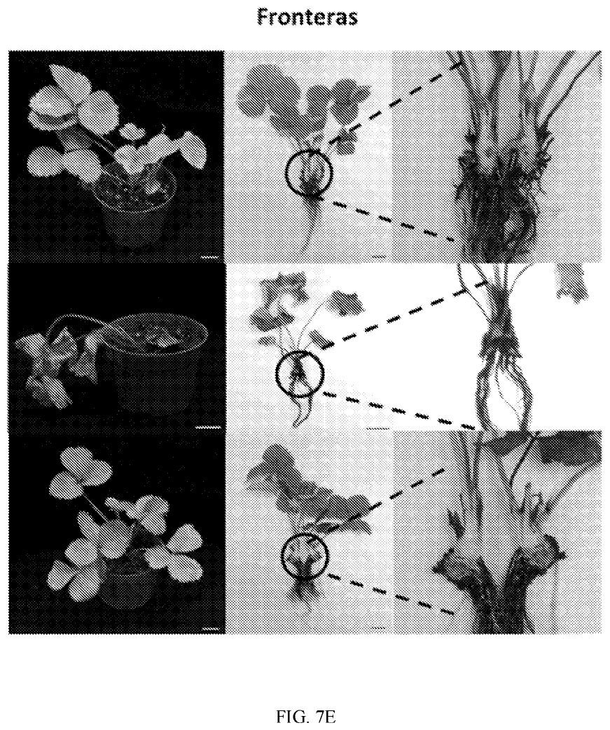

FIG. 8. Images illustrating phenotypic response of transient transformation assays to investigate the functional role of genes in strawberry resistance to P. cactorum pathogens. (A) Symptoms of the susceptible cultivar Florida Brilliance 17 days after inoculation with the P. cactorum pathogen. Plants transiently expressing pMDC32::empty vector were susceptible to P. cactorum infection and phytophthora crown rot. Plants transfected with pMDC32::FaWAK, and pMDC32::FaCNGC (overexpressing FaWAK and FaCNGC, respectively) exhibited decreased susceptibility to P. cactorum infection and phytophthora crown rot. (B) The phenotype of the resistant cultivar Florida Beauty 17 days after inoculation with the P. cactorum pathogen following transient gene silencing. After transient inoculation with pk7::empty vector, resistant plants exhibited resistance to P. cactorum infection and phytophthora crown rot. After transient inoculation with pk7::FaWAK and pk7::FaCNGC RNAi constructs, plants exhibited increased susceptibility to P. cactorum infection and phytophthora crown rot. Scale bar represents 2 cm.

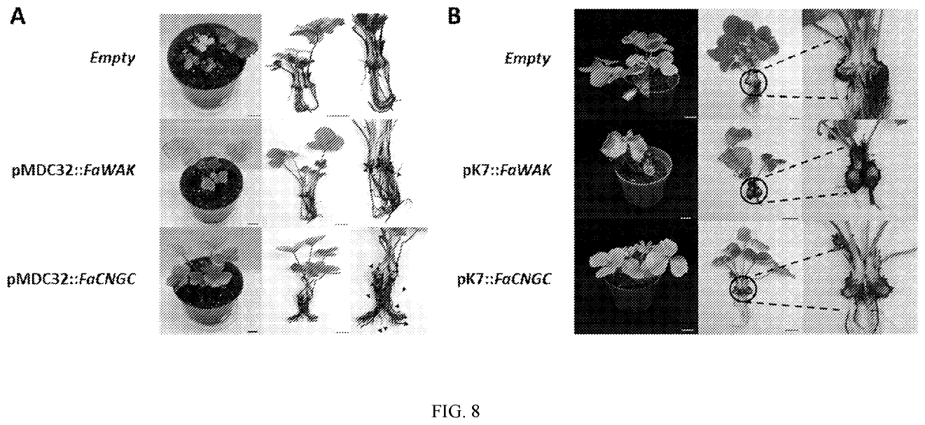

FIG. 9. Gene expression analysis of FaWAK and FaCNGC by qRT-PCR in strawberry resistance to P. cactorum pathogens. (A) The transient expression assay was conducted employing the pMDC32 overexpression vector to drive the expression of FaWAK and FaCNGC genes. (B) The transient expression assay was conducted employing the pK7GWIWG(II) RNAi vector to drive the knockdown expression of FaWAK and FaCNGC genes. The x-axis represents the vector composition used. EV denotes ‘Empty Vector, ‘Error bars represent standard deviation. Asterisks indicate significant outcomes with P values of Student's t-test (*P<0.05, **P<0.01, ***P<0.001, Student's t-test), respectively.

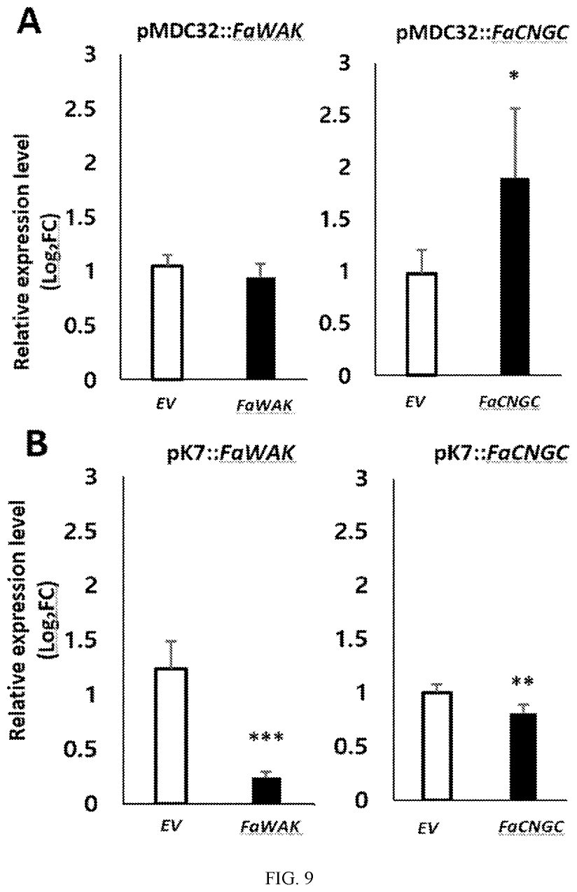

### DEFINITIONS

Unless otherwise defined, all terms of art, notations, and other scientific terminology used herein are intended to have the meanings commonly understood by those of skill in the art to which this invention pertains. Some techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Pena, ed., Humana Press, 1st edition, 2004); and, Agrobacterium Protocols (Wan, ed., Humana Press, 2nd edition. 2006). As appropriate, procedures involving the use of commercially available kits and reagents are generally carried out in accordance with manufacturer defined protocols and/or parameters unless otherwise noted.

The use of “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” indicates within an acceptable error range for the particular value as determined by one of ordinary skill in the art, which will depend in part on how the value is measured or determined, e.g., the limitations of the measurement system. For example. “about” can mean within 1 or more than 1 standard deviation, per the practice in the art. Alternatively, “about” can mean a range of 0 to 20%, 0 to 10%, 0 to 5%, or up to 1% of a given value. Where particular values are described in the application and claims, unless otherwise stated the term “about” means within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as well as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

The term “nucleic acid” refers to deoxyribonucleotides or ribonucleotides and polymers thereof (“polynucleotides”) in either single- or double-stranded form. Unless specifically limited, the term polynucleotide encompasses nucleic acids containing known analogues of natural nucleotides which have similar binding properties as the reference nucleic acid and are metabolized in a manner similar to naturally occurring nucleotides. Unless specifically limited, the term polynucleotide encompasses nucleic acids having one or more modified nucleotides. Modified nucleotides can modify binding properties or alter in vitro or in vivo stability. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The termnucleic acid is used interchangeably with gene, cDNA, and mRNA encoded by a gene.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted, and other elements added without sacrificing the necessary expression.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “plant” includes whole plants, plant organs (e.g., leaves, stems, flowers, roots, reproductive organs, embryos, and parts thereof, etc.), seedlings, seeds and plant cells, and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, (e.g., octoploid, diploid, haploid, and hemizygous).

The terms “elite breeding population” or “elite genotype” or “elite line” refer to specific genotypes of plants that have been bred by plant breeding programs to have similar essential alleles for desirable end use characteristics, agronomics, disease resistance, and adaptation in the target region in which they are to be grown.

The term “locus” refers to a position in the genome that corresponds to a measurable characteristic (e.g., a trait) or gene. A locus can be a genomic region or section of DNA (the locus) which correlates with a variation in a phenotype. A locus can comprise a single or multiple genes or other genetic information within a contiguous genomic region or linkage group.

A “marker” or “genetic marker” refers to a gene or nucleotide sequence that can be used to identify the presence or location of a trait determinant, locus, gene, and/or allele. A genetic marker may be described as a variation at a given genomic locus. A genetic marker may be a short DNA sequence, such as a sequence surrounding a single base-pair change (for example as in a single nucleotide polymorphism (SNP)), or a longer DNA sequence, for example, a microsatellite/simple sequence repeat (SSR)). A “marker allele” refers to the version of the marker that is present in a particular individual. A genetic marker can be used to identify individuals, genes, or loci in an off-spring originating from an individual parent. An “FaRPc2 marker” is a genetic marker that is linked, closely linked, tightly linked, or extremely tightly linked to the WAK1, CNGC1, and/or CNGC2 gene.

“Linked” refers to one or more genes or markers that are located within about 65 megabases (Mb) of one another on the same chromosome. Thus, two “linked” genes or markers may be separated, for example, by about 65 Mb, about 60 Mb, about 55 Mb, about 50 Mb, about 45 Mb, about 40 Mb, about 35 Mb, about 30 Mb, about 25 Mb, about 20 Mb, about 15 Mb, about 10 Mb, about 9.0. Mb, about 8.0 Mb, about 7.0 Mb, about 6.0 Mb, about 5.2 Mb, about 4.0 Mb, about 3.0 Mb, about 2.0 Mb, about 1.0 Mb, or fewer Mb.

“Closely linked” refers to one or more genes or markers that are located within about 2.0 Mb of one another on the same chromosome. Thus, two closely linked genes or markers may be separated, for example, by about 2.00 Mb, about 1.95 Mb, about 1.90 Mb, about 1.85 Mb, about 1.80 Mb, about 1.75 Mb, about 1.70 Mb, about 1.65 Mb, about 1.60 Mb, about 1.55 Mb, about 1.50 Mb, about 1.45 Mb, about 1.40 Mb, about 1.35 Mb, about 1.30 Mb, about 1.25 Mb, about 1.20 Mb, about 1.15 Mb, about 1.10 Mb, about 1.05 Mb, about 1.00 Mb, about 0.95 Mb, about 0.90 Mb, about 0.85 Mb, about 0.80 Mb, about 0.75 Mb, about 0.70 Mb, about 0.65 Mb, about 0.60 Mb, about 0.55 Mb, about 0.50 Mb, about 0.45 Mb, about 0.40 Mb, about 0.35 Mb, about 0.30 Mb, about 0.25 Mb, about 0.20 Mb, about 0.15 Mb, about 0.10 Mb, about 0.05 Mb, about 0.025 Mb, or about 0.01 Mb

“Tightly linked” refers to one or more genes or markers that are located within about 1.0 Mb of one another on the same chromosome. Thus, two tightly linked genes or markers may be separated, for example, by about 1.00 Mb, about 0.95 Mb, about 0.90 Mb, about 0.85 Mb, about 0.80 Mb, about 0.75 Mb, about 0.70 Mb, about 0.65 Mb, about 0.60 Mb, about 0.55 Mb about 0.5 Mb, about 0.45 Mb, about 0.4 Mb, about 0.35 Mb, about 0.3 Mb, about 0.25 Mb, about 0.2 Mb, about 0.15 Mb, about 0.1 Mb, or about 0.05 Mb.

“Extremely tightly linked” refers to one or more genes or markers that are located within about 100 kb of one another on the same chromosome. Thus, two extremely tightly linked genes or markers may be separated, for example, by about 100 kb, about 95 kb, about 90 kb, about 85 kb, about 80 kb, about 75 kb, about 70 kb, about 65 kb, about 60 kb, about 55 kb, about 50 kb, about 45 kb, about 40 kb, about 35 kb, about 30 kb, about 25 kb, about 20 kb, about 15 kb, about 10 kb, about 5 kb, or about 1 kb.

“Introgression” or “introgressing” of a locus means introduction of a locus from a donor plant comprising the locus of interest into a recipient plant by standard breeding techniques, wherein selection can be done phenotypically by means of observation of a plant characteristic including, but not limited to, characteristics such as the intemodal length or plant height, or selection can be done with the use of markers through marker-assisted breeding, or combinations of these. The process of introgressing is often referred to as “backcrossing” when the process is repeated two or more times. In introgressing or backcrossing, the “donor” parent refers to the parental plant with the desired gene or locus to be introgressed. The “recipient” parent (used one or more times) or “recurrent” parent (used two or more times) refers to the parental plant into which the gene or locus is being introgressed. Selection is started in the F1 or any further generation from a cross between the recipient plant and the donor plant, suitably by using markers as identified herein. The skilled person is, however, familiar with creating and using new molecular markers that can identify or are linked to a locus of interest.

A “homolog” or “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes are genes in different species that evolved from a common ancestral gene by speciation. Orthologs typically retain the same function in the course of evolution. “Paralogous” genes include genes related by duplication within a genome. Paralogs can evolve new functions in the course of evolution.

A “heterologous” sequence is a sequence which is not normally present in a cell, genome, or gene in the genetic context in which the sequence is currently found. A heterologous sequence can be a sequence derived from the same gene and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

Compositions or methods “comprising” or “including” one or more recited elements may include other elements not specifically recited. For example, a composition that “comprises” or “includes” a marker may contain the marker alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

The term “and/or” refers to and encompasses any and all possible combinations of one or more of the associated listed items, as well as the lack of combinations when interpreted in the alternative (“or”). The term “or” refers to any one member of a particular list and also includes any combination of members of that list.

The singular forms of the articles “a.” “an,” and “the” include plural references unless the context clearly dictates otherwise. For example, the term “a marker” or “at least one marker” can include a plurality of markers, including mixtures thereof.

The term “Phytophthora cactorum” or “P. cactorum” refers to a fungal-like plant pathogen belonging to the Oomycota phylum. Oospores of P. cactorum survive in the soil or in infected plants and, under optimal conditions, germinate and produce sporangia from which zoospores are released. Zoospores are motile and can disperse through water, entering root and crown tissues through wounds. After infection, young leaves turn bluish-green and wilt, followed by entire plant collapse and death.

The term “Phytophthora crown rot disease” or “PhCR” refers to the disease caused in strawberry by infection with P. cactorum (primarily) and P. nicotianae. PhCR is a disease of commercial importance worldwide, but particularly in annual strawberry production systems in California and Florida after the loss of methyl bromide soil fumigation.

The term “FaRPc2” refers to a large-effect quantitative trait locus for PhCR resistance (Fragaria Resistance to P. cactorum locus/gene 2) near 63cM on linkage group (LG) 7D, on chromosome 7-3, in octoploid strawberry. It is named as “locus/gene 2” since the first described resistance locus in Fragaria was identified on LG 6 of diploid F. vesca.

The terms “haplotype,” “H1,” “H2,” “H3,” and “H4” refer to a set of DNA variants found along a single chromosome that tend to be inherited together due to close proximity and, thus, rare occasions of recombination events occurring between them. These haplotypes may be associated with phenotypic characteristics, such as the presence of H1 and H4 haplotypes being associated with increased susceptibility to PhCR in strawberry, and H2 and H3 haplotypes being associated with increased resistance to PhCR in strawberry.

The term “accession” refers to a group of related plant material from a single species which is collected at one time from a specific location. Each accession is an attempt to capture the diversity present in a given population of plants. Accessions are given a unique identifier, an “accession number,” which is used to maintain associated information in the Germplasm Resources Information Network (GRIN) database.

The terms “high resolution melting” and “HRM” refer to the quantitative analysis of the melt curves of product DNA fragments following PCR amplification. HRM experiments generate DNA melt curve profiles that are both specific and sensitive enough to distinguish nucleic acid species based on small sequence differences, enabling mutation scanning, methylation analysis, and genotyping.

The term “trio binning” refers to a method of genome assembly that takes advantage of heterozygosity instead of trying to remove it. Most current technologies attempt to collapse parental haplotypes into a composite, haploid sequence, introducing erroneous duplications through mis-assembly of heterozygous sites as separate genomic regions. This problem is exacerbated in highly heterozygous genomes, particularly in cases such as allo-octoploid strawberry, resulting in fragmented and inflated assemblies that impede downstream analyses. In trio binning, a family trio is sequenced with short reads for both parents and long reads for an F1 offspring. Parent-specific k-mer markers are then identified from the parental reads and used to assign offspring reads into maternal and paternal bins before assembling each parental haploid genome separately. The ability of trio binning to accurately distinguish parental haplotypes increases at greater heterozygosity.

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

A “guide RNA” (gRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339: 823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT. NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

A “DNA donor template” is a nucleic acid, such as a single stranded DNA, linear double strand DNA, plasmid DNA or AAV sequence that provides the homology necessary for precise repair of a double-strand break. A DNA donor template contains two regions of homology, one region of homology to either side of the double strand break. A DNA donor template can also contain a heterologous sequence between the two regions of homology. Each region of homology can be about 30 to about 100 nucleotides in length.

A “regenerant” is a plant produced from a plant tissue cell, such as a genetically modified plant tissue cell.

### DETAILED DESCRIPTION

### I. Overview

Described are compositions for genetically modifying a strawberry plant to increase PhCR resistance of the plant. Described are compositions for modifying a Phytophthora crown rot disease (PhCR) resistance-related locus and/or one or more PhCR resistance-associated genes in a plant. Also described are methods of using the compositions for producing plants having a PhCR-resistant phenotype. In some embodiments, the plant is a Fragaria plant. A Fragaria plant can be any member of the Fragaria genus suitable for commercial strawberry production. A Fragaria plant can be a diploid species, a tetraploid species, a hexaploid species, an octoploid species, or any other ploidy species or hybrid thereof. A Fragaria plant can be, but is not limited to, Fragaria vesca, Fragaria x bifera, Fragaria x bringhurstii, Fragaria virginiana, Fragaria chiloensis, or Fragaria x ananassa. In some embodiments, the Fragaria plant is a Fragaria x ananassa plant.

A “PhCR resistance-associated locus” or “F. cactorum resistance-associated locus” comprises a locus that corresponds to the trait (phenotype) of measurably reduced PhCR disease severity incidence in a plant following infestation with P. cactorum. One such non-limiting example of a PhCR resistance-associated locus is the FaRPc2 locus. Plants with H2 and/or H3 haplotypes at the FaRPc2 locus exhibit the PhCR resistant phenotype, i.e., the plants have reduced severity of PhCR disease incidence compared to plants not carrying the H2 and/or H3 haplotype, with the strength of this effect highest for the H3 haplotype. Specific genes within the H3 haplotype of the FaRPc2 locus that correlate with PhCR resistance have been identified by comparison of elite breeding populations known to be PhCR resistant with other cultivars susceptible to PhCR. Such non-limiting examples of genes associated with PhCR resistance in plants include the WAK1, CNGC1, and/or CNGC2 genes.

A “FaRPc2 locus” comprises a polynucleotide encoding the Fragaria Resistance to Phytophthora cactorum locus/gene 2, at nucleotides 22,836,000 to 22,956,000 on chromosome 7-3. The FaRPc2 locus can include coding and non-coding sequence. Non-coding sequence includes, but is not limited to, regulator sequences, intron sequences and scaffold sequences. FaRPc2 marker is in a continuous nucleic acid region comprising an interval between base pairs 22,836,000 and 22,956,000 on chromosome 7-3 of the Fragaria plant.

A “WAK1 gene” comprises a polynucleotide encoding wall-associated receptor kinase 1. In some embodiments, a WAK1 gene encodes a protein having the amino acid sequence of SEQ ID NO: 2. In some embodiments, a WAK1 gene comprises the nucleotide sequence of SEQ ID NO: 1.

A “CNGC1 gene” comprises a polynucleotide encoding cyclic nucleotide-gated ion channel 1. In some embodiments, a CNGC1 gene encodes a protein having the amino acid sequence of SEQ ID NO: 4. In some embodiments, a CNGC1 gene comprises the nucleotide sequence of SEQ ID NO: 3.

A “CNGC2 gene” comprises a polynucleotide encoding cyclic nucleotide-gated ion channel 2. In some embodiments, a CNGC2 gene encodes a protein having the amino acid sequence of SEQ ID NO: 6. In some embodiments, a CNGC2 gene comprises the nucleotide sequence of SEQ ID NO: 5.

The WAK1, CNGC1, and CNGC2 genes (individually, in combinations of two or more, or collectively) may be referred to as “PhCR resistance-associated genes” or “P. cactorum resistance-associated genes.” The FaRPc2 locus may be referred to as “PhCR resistance-associated locus” or “P. cactorum resistance-associated locus.”

In some embodiments, constructs and systems for unaided homologous recombination, recombinase-based insertion, and DNA repair-based insertion targeted genetic modification of Fragaria plants are described. The constructs and systems can be used to insert a heterologous WAK1 gene, and heterologous CNGC1 gene, and/or a heterologous CNGC2 gene into a strawberry plant. The constructs and systems can also be used to replace an endogenous allele of WAK1, CNGC1, and/or CNGC2 that does not confer PhCR resistance with all or a portion of SEQ ID NO: 1, SEQ ID NO: 3, and/or SEQ ID NO: 5, respectively.

In some embodiments, nucleic acids for producing PhCR resistant plants using CRISPR systems are described. The CRISPR systems can target one or more of the PhCR resistance-associated genes in the FaRPc2 locus. The nucleic acids include, but are not limited to, nucleic acids comprising crRNAs or gRNAs and nucleic acids encoding crRNAs or gRNAs.

In some embodiments, nucleic acids for producing PhCR resistant plants using CRISPR systems are described. The CRISPR systems can be used to knock in one or more of a heterologous WAK1 gene, a heterologous CNGC1 gene, and a heterologous CNGC2 gene into a Fragaria plant cell. The nucleic acids include, but are not limited to, nucleic acids comprising crRNAs or gRNAs, nucleic acids encoding crRNAs or gRNAs, and DNA donor template nucleic acids. The CRISPR systems can knock in one or more of a heterologous WAK1 gene, a heterologous CNGC1 gene, and a heterologous CNGC2 gene unto the FaRPc2 locus of the plant genome or into an alternative site in the plant genome.

In some embodiments, nucleic acids for producing PhCR resistant plants using CRISPR systems are described. The CRISPR systems can be used to modify an endogenous WAK gene, an endogenous CNGC1 gene, and/or an endogenous CNGC2 gene in a Fragaria plant or plant cell. The nucleic acids include, but are not limited to, nucleic acids comprising crRNAs or gRNAs, nucleic acids encoding crRNAs or gRNAs, and DNA donor template nucleic acids. In some embodiments, endogenous WAK1 gene does not encode SEQ ID NO: 2 and the modified WAK1 gene encodes SEQ ID NO: 2. In some embodiments, endogenous CNGC1 gene does not encode SEQ ID NO: 4 and the modified CNGC1 gene encodes SEQ ID NO: 2. In some embodiments, endogenous CNGC2 gene does not encode SEQ ID NO: 6 and the modified CNGC1 gene encodes SEQ ID NO: 6.

In some embodiments, methods of producing PhCR resistant Fragaria plants and methods of genetically modifying a Fragaria plant to produce a PhCR resistant plant using a CRISPR system are described.

In some embodiments, Fragaria plants having a PhCR resistant phenotype produced using any one or more of the described CRISPR constructs are described.

The described FaRPc2 locus can be targeted to genetically modify Fragaria plants to yield a PhCR resistant phenotype. Fragaria plants containing an FaRPc2 H3 haplotype or one or more of WAK1, CNGC1, and CNGC2 genes exhibit reduced severity of PhCR disease incidence compared to the otherwise genetically identical plants. Fragaria plants lacking an FaRPc2 H3 haplotype or one or more of WAK1, CNGC1, and CNGC2 exhibit markedly increased severity of PhCR disease incidence compared to the otherwise genetically identical plants.

### II. Targeted Genetic Modification

A targeted genetic modification can comprise a targeted alteration to a polynucleotide of interest including, for example, a targeted alteration to a target genomic locus on chromosome 7-3 of a Fragaria plant, a targeted alteration to the FaRPc2 locus in a Fragaria plant, or a targeted alteration a genomic location suitable for expression of a heterologous gene in a Fragaria plant. Such targeted modifications include, but are not limited to, additions of one or more nucleotides, deletions of one or more nucleotides, substitutions of one or more nucleotides, a knockout of the polynucleotide of interest or a portion thereof, a knock-in of a polynucleotide of interest or a portion thereof (e.g., a heterologous WAK1 gene, coding sequence, or fragment thereof, a heterologous CNGC1 gene, coding sequence, or fragment thereof, and/or a heterologous CNGC2 gene, coding sequence, or fragment thereof), a replacement of an endogenous nucleic acid sequence with a heterologous nucleic acid sequence, insertion of a regulatory sequence upstream or downstream of an endogenous gene (e.g., a WAK1 gene, a CNGC1 gene, and/or a CNGC2 gene) or a combination thereof. In certain embodiments, at least 1, 2, 3, 4, 5, 7, 8, 9, 10 or more nucleotides are changed to form the targeted genomic modification. In some embodiments, the targeted genetic modification comprises a targeted DNA insertion of one or more polynucleotides encoding one or more PhCR resistance-associated genes.

Various methods can be used to generate the targeted modification in the polynucleotide or Fragaria plant genome of interest. Methods of obtaining the targeted genetic modifications as described in the instant application can comprise unaided homologous recombination, recombinase-based insertion, and DNA repair-based insertion and are known in the art (Dong et al., 2021, PNAS. 118(22) e2004834117). Recombinase-based insertion can comprise systems and constructs involving site-specific recombinases, including but not limited to, Cre:loxP systems, Flp:FRT systems, Dre:rox systems, VCre:loxVsystems, Gin:gix systems, Bxbl:attP/attB systems, phiC31:attP/attB systems. DNA repair-based insertion methods rely upon the activity of a nuclease agent.

The term “recognition site for a nuclease agent” includes a DNA sequence at which a nick or double-strand break is induced by a nuclease agent. The recognition site for a nuclease agent can be endogenous (or native) to the cell or the recognition site can be exogenous to the cell. In specific embodiments, the recognition site is exogenous to the cell and thereby is not naturally occurring in the genome of the cell. In still further embodiments, the recognition site is exogenous to the cell and to the polynucleotides of interest that one desires to be positioned at the target locus. In further embodiments, the exogenous or endogenous recognition site is present only once in the genome of the host cell. In specific embodiments, an endogenous or native site that occurs only once within the genome is identified. Such a site can then be used to design nuclease agents that will produce a nick or double-strand break at the endogenous recognition site.

The length of the recognition site can vary, and includes, for example, recognition sites that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

In one embodiment, each monomer of the nuclease agent recognizes a recognition site of at least 9 nucleotides. In other embodiments, the recognition site is from about 9 to about 12 nucleotides in length, from about 12 to about 15 nucleotides in length, from about 15 to about 18 nucleotides in length, or from about 18 to about 21 nucleotides in length, and any combination of such subranges (e.g., 9-18 nucleotides). It is recognized that a given nuclease agent can bind the recognition site and cleave that binding site or alternatively, the nuclease agent can bind to a sequence that is different from the recognition site. Moreover, the term recognition site comprises both the nuclease agent binding site and the nick/cleavage site irrespective whether the nick/cleavage site is within or outside the nuclease agent binding site. In another variation, the cleavage by the nuclease agent can occur at nucleotide positions immediately opposite each other to produce a blunt end cut or, in other cases, the incisions can be staggered to produce single-stranded overhangs, also called “sticky ends”, which can be either 5′ overhangs, or 3′ overhangs.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. Alternatively, a modified or engineered nuclease agent can be employed. An “engineered nuclease agent” includes a nuclease that is engineered (modified or derived) from its native form to specifically recognize and induce a nick or double-strand break in the desired recognition site. Thus, an engineered nuclease agent can be derived from a native, naturally occurring nuclease agent or it can be artificially created or synthesized. The modification of the nuclease agent can be as little as one amino acid in a protein cleavage agent or one nucleotide in a nucleic acid cleavage agent. In some embodiments, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA. Assays to measure the double-strand break of a recognition site by a nuclease agent are known in the art (e.g., TaqMan® qPCR assay, Frendewey D. et al., Methods in Enzymology, 2010, 476:295-307, which is incorporated by reference herein in its entirety).

In some embodiments, the recognition site is positioned within the polynucleotide encoding a selection marker. Such a position can be located within the coding region of the selection marker or within the regulatory regions, which influence the expression of the selection marker. Thus, a recognition site of the nuclease agent can be located in an intron of the selection marker, a promoter, an enhancer, a regulatory region, or any non-protein-coding region of the polynucleotide encoding the selection marker. In specific embodiments, a nick or double-strand break at the recognition site disrupts the activity of the selection marker. Methods to assay for the presence or absence of a functional selection marker are known.

In one embodiment, the nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See. WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference). In various embodiments. TAL effector nucleases are engineered that cut in or near a target nucleic acid sequence in, e.g., a locus of interest or a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In some embodiments, each monomer of the TALEN comprises 33-35 TAL repeats that recognize a single base pair via two hypervariable residues. In one embodiment, the nuclease agent is a chimeric protein comprising a TAL repeat-based DNA binding domain operably linked to an independent nuclease. In one embodiment, the independent nuclease is a FokI endonuclease. In one embodiment, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a spacer sequence of varying length (12-20 bp), and wherein the FokI nuclease subunits dimerize to create an active nuclease that makes a double strand break at a target sequence.

The nuclease agent employed in the various methods and compositions disclosed herein can further comprise a zinc-finger nuclease (ZFN). In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a FokI endonuclease. In one embodiment, the nuclease agent comprises a first ZFN and a second ZFN, wherein each of the first ZFN and the second ZFN is operably linked to a FokI nuclease subunit, wherein the first and the second ZFN recognize two contiguous target DNA sequences in each strand of the target DNA sequence separated by about 5-7 bp spacer, and wherein the FokI nuclease subunits dimerize to create an active nuclease to make a double strand break. See, for example, US20060246567; US20080182332; US20020081614; US20030021776; WO/2002/057308A2; US20130123484; US20100291048; WO/2011/017293A2; and Gaj et al. (2013) Trends in Biotechnology. 31(7):397-405 each of which is herein incorporated by reference.

In still another embodiment, the nuclease agent is a meganuclease. Meganucleases have been classified into four families based on conserved sequence motifs, the families are the LAGLIDADG. GIY-YIG, H-N-H, and His-Cys box families. These motifs participate in the coordination of metal ions and hydrolysis of phosphodiester bonds. Meganucleases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248: Lucas et al., (2001) Nucleic Acids Res 29:960-9; Jurica and Stoddard, (1999) Cell Mol Life Sci 55:1304-26: Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178: Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SceVI, I-SceVII, I-CeuI, I-CeuAIIP, I-Crel, I-CrepsbIP, I-CrepsbIIP, I-CrepsbIIIP, I-CrepsblVP, I-TliI, I-PpoI, PI-PspI, F-SceI, F-SceII, F-SuvI, F-TevI, F-TevII, I-AmaI, I-Anil, I-ChuI, I-CmoeI. I-CpaI, I-CpaII, I-CsmI, I-CvuI, I-CvuAIP, I-DdiI, I-DdiII, I-DirI. I-DmoI, I-HmuI. I-HmuII. I-HsNIP. I-LlaI, I-MsoI. I-NaaI. I-NanI. I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII. I-UarAP, I-UarHGPAIP, I-UarHGPAT3P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

Nuclease agents can further comprise restriction endonucleases, which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type Ius enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA 11, pp. 761-783, Eds. Craigie et al., (ASM Press, Washington. DC).

The nuclease agent employed in the various methods and compositions can also comprise a CRISPR/Cas system. Described are nucleic acids for producing PhCR resistant plants using a CRISPR (e.g., CRISPR/Cas) system. The described nucleic acids can be used to target modification of the FaRPc2 locus and/or to insert or express one or more PhCR resistance-associated genes in a plant.

A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g., operably linked to a promoter expressible in the plant). In some embodiments, the CRISPR system further includes a DNA donor template. In some embodiments, the CRISPR system can be delivered to a plant or plant cell via a bacterium. The bacterium can be, but is not limited to, Agrobacterium tumefaciens.

In some embodiments, the CRISPR system is designed to target one or more of the described PhCR resistance-associated genes and/or regions of the FaRPc2 locus. In some embodiments, the CRISPR system is designed knock in a heterologous WAK1 gene, coding sequence, or fragment thereof, a heterologous CNGC1 gene, coding sequence, or fragment thereof, and/or a heterologous CNGC2 gene, coding sequence, or a fragment thereof, into a locus in the Fragaria plant suitable for expression of a heterologous gene (transgene). The locus can be the FaRPc2 locus or another locus, In some embodiments, the CRISPR system is designed insert one or more a heterologous WAK1 gene, coding sequence, or fragment thereof, a heterologous CNGC1 gene, coding sequence, or fragment thereof, and/or a heterologous CNGC2 gene, coding sequence, or fragment thereof, into the genome or a Fragaria plant thereby resulting in increased expression of the heterologous WAK1 gene, coding sequence, or fragment thereof, the heterologous CNGC1 gene, coding sequence, or fragment thereof, and/or the heterologous CNGC2 gene, coding sequence, or fragment thereof, in the plant relative to a plant that does not contain the heterologous WAK1 gene, coding sequence, or fragment thereof, the heterologous CNGC1 gene, coding sequence, or fragment thereof, and/or the heterologous CNGC2 gene, coding sequence, or fragment thereof.

In some embodiments, the CRISPR system is designed to target one or more of the described PhCR resistance-associated genes and/or regions of the FaRPc2 locus. In some embodiments, the CRISPR system is designed insert one or more a heterologous WAK1 gene, coding sequence, or fragment thereof, a heterologous CNGC1 gene, coding sequence, or fragment thereof, and/or a heterologous CNGC2 gene, coding sequence, or fragment thereof, into the genome or a Fragaria plant thereby resulting in increased expression of the heterologous WAK1 gene, coding sequence, or fragment thereof, the heterologous CNGC1 gene, coding sequence, or fragment thereof, and/or the heterologous CNGC2 gene in the plant relative to a plant that does not contain the heterologous WAK1 gene, coding sequence, or fragment thereof, the heterologous CNGC1 gene, coding sequence, or fragment thereof, and/or the heterologous CNGC2 gene. In some embodiments, the CRISPR system is designed to modify the FaRPc2 locus to increase expression or activity of one or more of the endogenous WAK1, CNGC1, and/or CNGC2 genes.

The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

Guide sequences suitable for forming gRNAs or crRNAs for CRISPR system mediated genetic modification of a FaRPc2 locus are described. Suitable guide sequences include 17-20 nucleotide sequences along the FaRPc2 locus, the WAK1 genomic locus, the CNGC1 genomic locus, or the CNGC2 genomic locus, that are unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ in the FaRPc2 locus, the WAK1 genomic locus, the CNGC1 genomic locus, or the CNGC2 genomic locus can be used in forming a gRNA. In some embodiments, the guide sequence is 100% complementary to the target sequence. In some embodiments, the guide sequence is at least 90% or at least 95% complementary to the target sequence. In some embodiments, the guide sequence contains 0, 1, or 2 mismatches when hybridized to the target sequence. In some embodiments, a mismatch, if present, is located distal to the PAM, in the 5′ end of the guide sequence.

Additional guide sequence suitable for forming gRNAs or crRNAs for CRISPR system mediated genetic modification of Fragaria plants include 17-20 nucleotide sequences in the Fragaria genome that are unique compared to the rest of the genome and immediately adjacent (5′) to a protospacer-adjacent motif (PAM) site and suitable for insertion of a heterologous gene. Such sites are term safe harbor sites.

The DNA donor template contains sequence to be inserted into the genome of the plant. In some embodiments, the DNA donor template to be used with a CRISPR system comprises sequences for a heterologous WAK1 gene, coding sequence, or fragment thereof, a heterologous CNGC1 gene, coding sequence, or fragment thereof, and/or a heterologous CNGC2 gene, coding sequence, or fragment thereof (e.g., SEQ ID NOs: 1, 3, and/or 5, respectively). The donor template can further comprise one or more regulator sequence operatively linked to the coding sequence that drive expression of the coding sequence in the plant cell. In some embodiments, the DNA donor template to be used with a CRISPR system comprises sequence for modifying the endogenous WAK1. CNGC1, and/or CNGC2 genes to increase expression of activity of the endogenous WAK1, CNGC1, and/or CNGC2 genes.

DNA donor templates further comprise 5′ and 3′ homology regions located 5′ and 3′ to the sequence to be inserted into the genome. The homology regions comprise about 30 to about 100 nucleotides that are complementary to a corresponding number of nucleotides in the genome on either side of the double strand break created by the CRISPR nuclease.

The DNA donor template can be provided a single strand DNA, double strand DNA, plasmid DNA, or adeno-associated vector DNA.

It is understood that RNA equivalents of any listed DNA sequences, substituting uracils (U) for thymines (T), may be used. An “RNA equivalent” is an RNA molecule having essentially the same complementary base pair hybridization properties as the listed DNA sequence.

CRISPR modification of a PhCR resistance locus or other genomic site is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type 1I endonuclease derived from S. pyogenes utilizes an NGG PAM sequence located on the immediate 3′ end of the guide sequence. Other PAM sequences include, but are not limited to, NNNNGATT (Neisseria meningitidis). NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

Two or more guide RNAs can used with the same RNA-guided DNA endonuclease (e.g., Cas nuclease) or different RNA-guided DNA endonucleases.

In some embodiments, two or more gRNAs targeting the insertion of two or more different PhCR resistance-associated genes are used. The two or more gRNAs can be used with the same RNA-guided DNA endonuclease or different RNA-guided DNA endonucleases.

In some embodiments, three or more gRNAs targeting the insertion of three or more different PhCR resistance-associated genes are used. The three or more gRNAs can used with the same RNA-guided DNA endonuclease or different RNA-guided DNA endonucleases.

Guide RNAs for modification of PhCR resistance-associated loci in other Fragaria plants and other plants susceptible to P. cactorum infestation, including but not limited to such economically important plants as apple, pear, stone fruits (e.g., peaches, plums, etc.), and ornamentals are generated in a similar manner by identifying the corresponding ortholog sequences of the PhCR resistance-associated loci and/or genes and selecting target sequences as described above.

Any of the above-described guide RNAs can be provided as an RNA or a DNA encoding the RNA.

In some embodiments, a CRISPR system comprises one or more guide RNAs and a nucleic acid encoding an RNA-guided DNA endonuclease. In some embodiments, a CRISPR system comprises one or more guide RNAs, a nucleic acid encoding an RNA-guided DNA endonuclease, and a DNA donor template.

In some embodiments, a CRISPR system comprises one or more guide RNAs and a one or more nucleic acids encoding two or more different RNA-guided DNA endonucleases. In some embodiments, a CRISPR system comprises one or more guide RNAs, one or more nucleic acids encoding two or more different RNA-guided DNA endonucleases, and at least one DNA donor template.

In some embodiments, a CRISPR system comprises a guide RNA and an RNA-guided DNA endonuclease in a complex. In some embodiments, a CRISPR system comprises a guide two or more RNAs each in a complex with an RNA-guided DNA endonuclease. In some embodiments, a CRISPR system comprises a guide RNA and an RNA-guided DNA endonuclease in a complex and a DNA donor template.

### III. Modified Plants

Methods of producing PhCR resistant plants and methods of genetically modifying a plant to produce a PhCR resistant plant are described. In some embodiments, the plants are modified using a CRISPR system. Other methods known in the art may also be used to produce modified plants (e.g., Fragaria plants) to insert a heterologous WAK1 gene, coding sequence, or fragment thereof, a heterologous CNGC1 gene, coding sequence, or fragment thereof, and/or a heterologous CNGC2 gene, coding sequence, or fragment thereof, or to modify an endogenous WAK1 gene, an endogenous CNGC1 gene, and/or an endogenous CNGC2 gene.

Described are methods of generating genetically modified PhCR resistant plants comprising introducing into a plant, a plant tissue, or a plant cell, one or more of the described CRISPR systems. In some embodiments, genetically modified PhCR resistant plants created using a CRISPR system are described. In some embodiments, the CRISPR system is a CRISPR/Cas system.

In some embodiments, methods are described for producing a PhCR resistant strawberry plant, the methods comprising the step of introducing into the plant one or more of the described CRISPR systems.

Nucleic acids may be introduced into a plant cell or cells using a number of methods known in the art, including but not limited to electroporation, DNA bombardment or biolistic approaches, microinjection, via the use of various DNA-based vectors such as Agrobacterium tumefaciens and Agrobacterium rhizogenes vectors, and CRISPR or CRISPR/Cas9. Once a plant cell has been successfully transformed, it may be cultivated to regenerate a transgenic plant (regenerant).

Various methods for introducing the transgene expression vector constructs of the invention into a plant or plant cell are well known to those skilled in the art, and any method capable of transforming the target plant or plant cell may be utilized.

In some embodiments, Agrobacterium tumefaciens is used to deliver CRISPR system nucleic acids to a plant. Agrobacterium-mediated transformation of a large number of plants are extensively described in the literature (see, for example, Agrobacterium Protocols, Wan, ed., Humana Press. 2^{nd} edition, 2006). Various methods for introducing DNA into Agrobacteria are known, including electroporation, freeze/thaw methods, and triparental mating. In some embodiments, a pMON316-based vector is used in the leaf disc transformation system of Horsch et al. Other commonly used transformation methods include, but are not limited to, microprojectile bombardment, biolistic transformation, and protoplast transformation of naked DNA by calcium, polyethylene glycol (PEG) or electroporation (Paszkowski et al., 1984, EMBO J. 3: 2727-2722; Potrykus et al., 1985, Mol. Gen. Genet. 199: 169-177; Fromm et al., 1985, Proc. Nat. Acad. Sci. USA 82: 5824-5828; Shimamoto et al., 1989, Nature, 338: 274-276.

To transgenic plants may be used to generate subsequent generations (e.g., T₁, T2, etc.) by selfing of primary or secondary transformants, or by sexual crossing of primary or secondary transformants with other plants (transformed or untransformed).

The described CRISPR systems can be used to genetically modify- or introduce one or more PhCR resistance-associated genes in a plant. The plant can be a plant having another trait of interest. Delivery of the CRISPR system leads to insertions near the target sequence, resulting in expression of PhCR resistance-associated genes in the plant. Introducing a PhCR resistant phenotype into a plant having a desired trait, using the described CRISPR systems and methods, may result in a cost savings for plant developers because such methods eliminate traditional plant breeding.

In some embodiments, the described CRISPR systems can be used to genetically modify or introduce 1, 2, 3, or more PhCR resistance-associated genes in a plant.

In some embodiments, the CIRSPR system is used to modify or introduce one or more PhCR resistance-associated loci into a transgenic strawberry line. The transgenic strawberry line can also contain one or more genes for herbicide tolerance, increased yield, insect control, other fungal disease resistance, virus resistance, bacterial disease resistance, germination and/or seedling growth control, enhanced animal and/or human nutrition, improved processing traits, or improved flavor, among others.

Plants produced using the described CRISPR systems (having one of more of a WAK1 gene, coding sequence of fragment thereof, a CNGC1 gene, coding sequence of fragment thereof, and a CNGC2 gene, coding sequence of fragment thereof, and/or other PhCR resistance-associated genes and/or PhCR resistance-associated loci) have a PhCR resistant phenotype. The PhCR resistant plants can produce similar sizes and quantities of fruit to an otherwise genetically similar plants lacking the genes and/or loci that confer resistance to P. cactorum and PhCR. In some embodiments, the PhCR resistant plants produce fruits at a yield of greater than 50%, greater than 60%, greater than 70%, greater than 80%, greater than 90% of the yield of an otherwise genetically similar plant lacking the genes and/or loci that confer resistance to P. cactorum and PhCR when grown under the same conditions. In some embodiments, the PhCR resistant plants produce fruits at a higher yield than an otherwise genetically similar plant lacking the genes and/or loci that confer resistance to P. cactorum and PhCR when grown under the same conditions. In some embodiments, the PhCR resistant plants produce fruits having an average size that is greater than 50%, greater than 60%, greater than 70%, greater than 80%, or greater than 90% of the average size of fruits produced by an otherwise genetically similar plant lacking the genes and/or loci that confer resistance to P. cactorum and PhCR when grown under the same conditions. In some embodiments, the PhCR resistant plants produce fruits having an average size that is greater than an otherwise genetically similar plant lacking the genes and/or loci that confer resistance to P. cactorum and PhCR when grown under the same conditions. In some embodiments, the PhCR resistant plants produce fruits having an average weight that is greater than 50%, greater than 60%, greater than 70%, greater than 80%, or greater than 90% of the average weight of fruits produced by an otherwise genetically similar plant lacking the genes and/or loci that confer resistance to P. cactorum and PhCR when grown under the same conditions. In some embodiments, the PhCR resistant plants produce fruits having an average weight that is greater than an otherwise genetically similar plant lacking the genes and/or loci that confer resistance to P. cactorum and PhCR when grown under the same conditions. In some embodiments, the PhCR resistant plants have decrease incidence or severity of PhCR compared to an otherwise genetically similar plant lacking the genes and/or loci that confer resistance to P. cactorum and PhCR when grown under the same conditions.

### IV. Detection of a Modified Gene

Modification of a FaRPc2 locus can be detected or confirmed by any means known in the art for detecting genetic modifications in plants. Modification of a Fragaria plant to insert a heterologous WAK1 gene, coding sequence or fragment thereof, a heterologous CNGC1 gene, coding sequence or fragment thereof, and/or a heterologous CNGC2 gene, coding sequence or fragment thereof, can be detected or confirmed by any means known in the art for detecting genetic modifications.

In some embodiments, a modification can be detected in a genomic DNA sample. Genomic DNA samples include, but are not limited to, genomic DNA isolated directly from a plant, cloned genomic DNA, or amplified genomic DNA.

Genetic analysis methods include, but are not limited to, polymerase chain reaction (PCR)-based detection methods (for example, TaqMan assays), microarray methods, mass spectrometry-based methods and/or nucleic acid sequencing methods, including whole genome sequencing. In some embodiments, the detection of genetic modification in a sample of DNA, RNA, or cDNA may be facilitated through the use of nucleic acid amplification methods. Such methods specifically increase the concentration of polynucleotides that span a target site, or include that site and sequences located either distal or proximal to it. Such amplified molecules can be readily detected by gel electrophoresis, fluorescence detection methods, or other means.

In some embodiments, a FaRPc2 locus genetic modification or insertion of a heterologous WAK1 gene, coding sequence, or fragment thereof, a heterologous CNGC1 gene, coding sequence, or fragment thereof, and/or a heterologous CNGC2 gene, coding sequence, or fragment thereof, is detected by hybridization to allele-specific oligonucleotide (ASO) probes. ASO probes are disclosed in U.S. Pat. Nos. 5,468,613 and 5,217,863. Single or multiple nucleotide variations in nucleic acid sequence can be detected in nucleic acids by a process in which the sequence containing the nucleotide variation is amplified, spotted on a membrane, and treated with a labeled allele-specific oligonucleotide probe.

In some embodiments, a FaRPc2 locus genetic modification or insertion of a heterologous WAK1 gene, coding sequence, or fragment thereof, a heterologous CNGC1 gene, coding sequence, or fragment thereof, and/or a heterologous CNGC2 gene, coding sequence, or fragment thereof, is detected by probe ligation methods. Probe ligation methods disclosed in U.S. Pat. No. 5,800,944 where sequence of interest is amplified and hybridized to probes followed by ligation to detect a labeled part of the probe.

In some embodiments, microarrays can be used for detection of FaRPc2 locus genetic modification or insertion of a heterologous WAK1 gene, coding sequence, or fragment thereof, a heterologous CNGC1 gene, coding sequence, or fragment thereof, and/or a heterologous CNGC2 gene, coding sequence, or fragment thereof. For microarray detection, oligonucleotide probe sets are assembled in an overlapping fashion to represent a single sequence such that a difference in the target sequence at one point would result in partial probe hybridization (Borevitz et al., Genome Res. 13:513-523, 2003: Cui et al., Bioinformatics 21:3852-3858, 2005). Typing of target sequences by microarray-based methods is disclosed in U.S. Pat. Nos. 6,799,122; 6,913,879; and 6,996,476.

In some embodiments, a FaRPc2 locus genetic modification or insertion of a heterologous WAK1 gene, coding sequence, or fragment thereof, a heterologous CNGC1 gene, coding sequence, or fragment thereof, and/or a heterologous CNGC2 gene coding sequence, or fragment thereof, can be directly identified or sequenced using nucleic acid sequencing technologies. Methods for nucleic acid sequencing are known in the art and include technologies provided by 454 Life Sciences (Branford, Conn.). Agencourt Bioscience (Beverly, Mass.), Applied Biosystems (Foster City, Calif), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays.

In some embodiments, the presence of a FaRPc2 marker (e.g., a WAK1 gene marker, a CNGC1 gene marker, and/or a CNGC2 gene marker) in a plant may be detected through the use of a nucleotide probe. A probe may be, but is not limited to, nucleotide molecule, polynucleotide, oligonucleotide, DNA molecule, RNA molecule, PNA, UNA, locked nucleotide, or modified polynucleotide. Polynucleotides can be synthesized by any means known in the art. A probe may contain all or a portion of the nucleotide sequence of the genetic marker and optionally, one or more additional sequences. The one or more additional sequences can be contiguous nucleotide sequence from the plant genome, non-contiguous nucleotide sequence from the plant genome, or sequence that is not from the plant genome. Additional, contiguous nucleotide sequence can be “upstream” or “downstream” of the original marker, depending on whether the contiguous nucleotide sequence from the plant chromosome is on the 5′ or the 3′ side of the original marker, as conventionally understood. As is recognized by those of ordinary skill in the art, the process of obtaining additional, contiguous nucleotide sequence for inclusion in a marker may be repeated nearly indefinitely (limited only by the length of the chromosome), thereby identifying additional markers along the chromosome.

A polynucleotide probe may be labeled or unlabeled. A wide variety of techniques are readily available in the art for labeling a nucleotide probe. Nucleotide labels include, but are not limited to, radiolabeling, fluorophores, haptens, antibodies, antigens, enzymes, enzyme substrates, enzyme cofactors, and enzyme inhibitors. A label may provide a detectable signal by itself (e.g., a radiolabel or fluorophore) or in conjunction with other agents.

A probe may be an exact copy of a marker to be detected. A probe may also be a nucleic acid molecule comprising, or consisting of, a nucleotide sequence which is substantially identical to an inserted nucleic acid sequence. The term “substantially identical” may refer to nucleotide sequences that are more than 85% identical. For example, a substantially identical nucleotide sequence may be 86%, 87%, 88%, 89%, 90%, 91%, 92%, 93%, 94%, 95%, 96%, 97%, 98%, or 99% identical to the reference sequence.

A probe may also be a nucleic acid molecule that is “specifically hybridizable” or “specifically complementary” to an exact copy of the marker to be detected (“DNA target”). “Specifically hybridizable” and “specifically complementary” are terms that indicate a sufficient degree of complementarity such that stable and specific binding occurs between the nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired. Thus, an oligonucleotide probe is “specifically hybridizable” to a marker allele if stable and specific binding occurs between the oligonucleotide probe and the marker allele (e.g., a SNP marker) under stringent hybridization conditions, but stable and specific binding does not occur between the oligonucleotide probe and the wild-type allele at the marker position.

In some embodiments, a probe comprises a pair of primers designed to produce an amplification product, wherein the amplification product is directly or indirectly determinative for the presence or absence of a PhCR resistance marker.

### V. Marker-Assisted Selection

Described are markers and methods for marker-assisted selection (MAS) of the FaRPc2 locus. Use of MAS in FaRPc2 breeding programs can be used to facilitate incorporation of the locus into diverse genetic backgrounds.

Described is a locus responsible for the PhCR resistance in Fragaria plants (strawberries). The FaRPc2 locus maps to a 1120-kb interval between bp 22,836,000-23,956,000 bp on chromosome 7-3.

FaRPc2 markers useful for genotyping (mapping, tracking, identifying, analyzing) a FaRPc2 locus in a Fragaria plants are described. In some embodiments, a FaRPc2 marker comprises a detectable genetic marker linked, closely linked, tightly linked, or extremely tightly to a FaRPc2 locus. In some embodiments, a FaRPc2 marker comprises a detectable genetic marker linked, closely linked, tightly linked, or extremely tightly linked to a genomic sequence encompassed by base pairs 22,836,000-23,956.000 bp on chromosome 7-3 of a Fragaria plant. In some embodiments, a FaRPc2 marker is a detectable genetic marker linked, closely linked, tightly linked, or extremely tightly linked to one or more of: SEQ ID NO: 1, SEQ ID NO: 3 and/or SEQ ID NO: 5.

In some embodiments, a FaRPc2 marker comprises a PCR amplification product, a single nucleotide polymorphism (SNP), a restriction fragment length polymorphism (RFLP), an amplified fragment length polymorphism (AFLP), a simple sequence repeat (SSR), a simple sequence length polymorphism (SSLP), an insertion/deletion polymorphism (indel), a variable number tandem repeat (VNTRs), or a random amplified polymorphic DNA (RAPD) linked, closely linked, tightly linked, or extremely tightly linked to one or more of: SEQ ID NO: 1, SEQ ID NO: 3 and/or SEQ ID NO: 5.

With the described FaRPc2 markers, PhCR resistant plants can be rapidly and efficiently identified. The identification of the FaRPc2 resistance locus can be used to aid in introgressing the PhCR resistance trait into strawberry plants.

In some embodiments, the FaRPc2 markers may be used in marker-assisted selection to produce PhCR resistant strawberry plants and/or introgress a PhCR resistance trait from donor strawberry plants to recipient plants strawberry plants are described.

In some embodiments, the described FaRPc2 markers may be used in marker-assisted selection to transfer (introgress) segment(s) of DNA that contain one or more determinants of PhCR resistance.

### BRIEF DESCRIPTION OF THE SEQUENCES

The nucleotide and amino acid sequences listed in the accompanying sequence listing are shown using standard letter abbreviations for nucleotide bases, and single-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 1
Sequences

WAK1 cDNA sequence (SEQ ID NO: 1)
ATGGCCTTACATGGGAGAATGCTTCTTTTGCAACTCTCTTTGGCCGCAGTGCTATTAGCAACGACAATGCTAG
CAGCTGCTCAAGCCTCACCGCCTGTCTGCAATGAGAGCTGCGGTGGTGTCTCAGTTCCATATCCATTTGGTCT
CACTGATGGTTGTTACCTACATGTGCCGGGACAAGATTCTCAGCAGCCATTCAAGATCACTTGCAACACCACC
ACCTCGCAACCATCCCTACAGTTCTCGGACAGCGATAACTTCCCCACAAATATTGCCAACATTTTTTTGGAAG
AGAGTGCGCTGCAAGTAATGATGGCCCCGTCCTATAACTGCTACACCAACAATACATACAACCCCTCCATGGA
CAAAGTCATGGACCTCAATCTTCCGCCTTCTTACACCCTCTCCAACAGAAACAAGGTTTATAACCTTGGGTGC
AACAAAGTAACGAAACTCATAGGTTATCCTCTTGAAGTTACAGACCCTGAGCAGCTCTTTCAAGTACGGTTCA
GCGGTGTAAGCTTGTGCCAAGATGAATTCGGCAAGAGATTCCCCGACACTTGCACCGGATTCGGGTGCTCCGT
AAATTCTATCCCTAGCGGACTGCAGAATATTACGGTGGATGTGTGGACACTCGGTACCGGCCTCGGAAAAACG
GAGCAGTGGGGACTGAGCTACCCTTGCAGTTACGGCTTCGTTGTGGATGAACGCAACTTCACATTCGCCGGGA
ATCAAAGTTTTGTTGCACTGAGGTCCGGCGCAATAAAGAAGCTTCCGGTGCTTGCTAATTGGGCGATTGGGAA
TGACAACTGCGAAGCAGCAAAGAAGAAGAACGAGACTGCTTTTGCATGCAAGACTGTGAATTCCAAGTGTGTC
GACCGTGCTGGTGGTGGTTACTTTTGCCAGTGTGAGGATGGTTACGAAGGGAACCCATACCTCCCCGATAATT
GCCTAGATATCAATGAGTGCAAGCATTCAACCCTTTGCAGTGGTCCTGCAACTTGCATAAACTCAATTGGAAG
TTACACCTGTAAATGTCACAAGGGCCATAGAAATGATGACCACGACAAGAATAAGTGTGTCCTAATCACTGAA
ACCTCCAGCAAAAATGACAAAGAAATGAAAATTTCCCTGGGTGTCTCTTTGACCTTCCTAGTCATACTGATTA
TAACTTTTTCGATATACTGCGTACTGAAAAGAAGAAAGTTCAAGCATCTCTGTGACAGGTACTACAAAGACAA
TGGGGGCTTCTTGTTACCACAAGAAATGCAGAAGTACAAAGGGTCTCAGGCACCCAGAATCTTTAAATTAGAA
GAGCTTAACAAGGCAACCAACAAGTTTGATCCCCATGAAATAATTGGAGAAGGAGGCTTTGGATTGGTTTACA
AGGGAACATTGCCGGACAACAAGCAGGAGGTTGCCATAAAGAAGTCAAAAACTGATGCTCCCACCATGACCCC
CGAAAGTAGGATCGCCCACACTAAGCAGTTCATTAATGAGATGATTGTTCTCTCTGGAATCAAGCATAGAAAT
GTCGTGAGGCTCTTAGGTTGTTGTTTGGAGACCAAAACGCCTATATTAGTGTACGAGTTTGTCTGCAATGGCA
CACTTTATGAGCACATTCATAAACAGAAAGGCAAAGGACCTCTTTCCTTTAGGCAACGAGTGAAGATAGCAGC
AGAAACAGCAGGATCCCTAGCATACTTGCACTACAACGCTTCTTCCACGCCAATTCTGCATCGAGACGTAAAG
GCATCTAACATCCTACTGGATGAGAATTGCACAGCCAAAATATCAGACTTTGGAGCTTCAAAATTGGTTCCCG
AAGATGAAAACACTCAACTGGCTACTTTGGTGCAAGGGACGCTAGGGTACTTGGATCCTCAATATATTCAAAC
ACACACACTGACGGAGAAGAGTGATGTCTACAGTTTCGGGGTTGTCCTTGTGGAACTGATAACGAGTCAAGCG
GCAATTAATTCTAACAAGAAGCTTGAGGCAGAGAAAAACCTAGCCAATTGTTTTTTAAAGTCGGTCGAAGATA
ATCACTTGGATCAGATTCTTGATCATGAAATTATCAAAGATAGCTTAGAAATAGCTGAACAAGTAGCCCATCT
CGCCAAAAGATGTTTGAGTTTGAAAGGAGATGATAGGCCTGCCATGAGAGAAGTAGAAACGGAGCTAGTGGCA
ATATTGGCAGTTATGGAAAAGCGTCCCGGAGGAAAGCCTGACTCCTCCCCCAAAGAGACCGATTACTTGCTTG
CAGCGTCACCTTCAAATGCTTTCGTTGTTGATGTTAGAAGTGATGAAGGTGAACTCACAACCAGCATAGATTA
TGACCAGAGCATGCAGAATCAATCCCAGATGACGAGGCCTTATGATAGTGGGAGATAG
WAK1 amino acid sequence (SEQ ID NO: 2)
MALHGRMLLLQLSLAAVLLATTMLAAAQASPPVCNESCGGVSVPYPFGLTDGCYLHVPGQDSQQPFKITCNTT
TSQPSLQFSDSDNFPTNIANIFLEESALQVMMAPSYNCYTNNTYNPSMDKVMDLNLPPSYTLSNRNKVYNLGC
NKVTKLIGYPLEVTDPEQLFQVRFSGVSLCQDEFGKRFPDTCTGFGCSVNSIPSGLQNITVDVWTLGTGLGKT
EQWGLSYPCSYGFVVDERNFTFAGNQSFVALRSGAIKKLPVLANWAIGNDNCEAAKKKNETAFACKTVNSKCV
DRAGGGYFCQCEDGYEGNPYLPDNCLDINECKHSTLCSGPATCINSIGSYTCKCHKGHRNDDHDKNKCVLITE
TSSKNDKEMKISLGVSLTFLVILIITFSIYCVLKRRKFKHLCDRYYKDNGGFLLPQEMQKYKGSQAPRIFKLE
ELNKATNKFDPHEIIGEGGFGLVYKGTLPDNKQEVAIKKSKTDAPTMTPESRIAHTKQFINEMIVLSGIKHRN
VVRLLGCCLETKTPILVYEFVCNGTLYEHIHKQKGKGPLSFRQRVKIAAETAGSLAYLHYNASSTPILHRDVK
ASNILLDENCTAKISDFGASKLVPEDENTQLATLVQGTLGYLDPQYIQTHTLTEKSDVYSFGVVLVELITSQA
AINSNKKLEAEKNLANCELKSVEDNHLDQILDHEIIKDSLEIAEQVAHLAKRCLSLKGDDRPAMREVETELVA
ILAVMEKRPGGKPDSSPKETDYLLAAS PSNAFVVDVRSDEGELTTSIDYDQSMQNQSQMTRPYDSGR*
CNGC1 cDNA sequence (SEQ ID NO: 3)
ATGGCTAAACATCAGGGACCAAACAATGAAATCCAAATGAGCATTACACCAGATGTGTCTTATGATTTCCATA
GTCCTGAGGCTCTAAAACCGACACGCGAACATGGACAACCTCAAGCAAGCAGCAAAAGATGGAATTGGCCAGC
AGTTGCCGGAGTTTCTTCAGCTTTGAACCCATGGTGGAATAAGATACTGATAACTTCATGTGTGATTGCAGTT
TTTTTTGATCCCTTGTTCTTTTACATTCCATACACCAGCGAGGAAAAAAAGTGTATGGGAAACGACGAAAGAG
TGCAGACTCCAGCTCTGATTTTCCGATCAGTCACAGACATCATTTTCCTGGTGCATATCATATACCAACTATA
TGTGGCTATCAAATTTGCACACTCAAGGGTTTCCGAGGATGAAAGTTTGGTTTTTAGATATGAATCGGTATCC
CGAGAGCAAATGATTCAGTTTGCGAAGGCATTTTCTCACAAGTTGTCATGGCGATCTTTCCTAACTGACGTTT
TCGCTATTTTGCCCATACCACAACTTCTAATAGTAGGATTGTTTTTCAACATCGGAGGCAATAACTACTTGTT
AAAAAGAAAGATCGTGGGCGGTTTTCTTCTAATCCAATATGTGCCAAGAATCTATCGAATTTATCTTTCATCT
GTCGCGGTCACAGAATCTGCATTGTGGGTTAGAGGTGTATTTAACTTCTTCCTCTACATTCTTGCGAGTCATG
CACTTGGAGCCTTTTGGTACTTTTGTTCTATTCAGCGAGAGACATCATGTTGGTATCGAACTTGCGTCGCTAC
AAATATATCAGAATCAAAATGCACCTTTTACTGCCATAACAGTACTGCTGCACTTATAACCCCACAATTCAAG
ACAACTCTAGACGAGAAATGCATTCTAAAAGTTCCATATAACATGACCGATCCACCATTTGATTTTGGAATAT
TTTTTGATGCCCTCAAAAATGATATCCAAGGGAAAATAAATGTTCCACAAAAGATTGGTTACTGTTTTTGGTG
GGGCTTGCGAAACTTGAGTAATTTTGGTACAAGTTTGGAAACAAGTACCTATCTGTGGGAAAACAGCTTTGCC
ATTTTAATTTCTATCATCGGCTTGCTGTTGTTTTTATACCTCATTGGAAATGTACAGATTTATATGCAGAGGG
CAACTACCAGAGCGGAGGAGGTAAGAGAGAAGCTTCGGATCAAAAAGAACGATATAGATAATTGGGTCCAAAA
TAATGGTATCGAGAAGGACATGAAGGAAGAGATCATGAAAAACATCGTTGAAAAGTTGGAAAACGACATAAGT
GCAGATCTGGATGATATATACTCTATTCTTCCCCCGTATACCAGGAAAGTTGTGAAGCGATTTGTCGGCATGA
GGACGCTAAGAAATGTACCAATGCTTAGCCAGGTGGATGGAAGAGTTTTGAAGATGATGTGCGACTATCTGAA
GCCGGTCAAGTACCAGGAGAATACAATGGTTTTTGAAACGGGAGTACCATTTGATAGAATGGTCTTCATTACA
GATGGGTCAATGTGGACTTACACGATTGCTACTGATCATCAGACTAGTCATTCTGGAAAAGCAACGTCAGGAG
GACTAGTTACTTCTTCGTCCACGGAAGCTCACTTCCTCAAGAAGGGTGATGCTTATGGACACATGCTTCTGCA
ATTAGCATCATCATCTTCTACGGCGGTTCCTATCTCAGTTGCAAATGTCAGGTGCCACACAAAGGTAGAGGCG
TTTGTCCTCATGGCCAATGACTTGAGAAACATAGTAACTGCGTGCGGAGATTTGTGGCCGGCTTTTGACTACA
AGGCTTCTCAAGATCAGGCGGCTGATCAGATGGCACCGCCGGGGCATTTTCAGCAACAACAAGGGCCAAAGAA
GCGTACTGTAGCATCATCAAATATCCATCCTACCGGCTACAGTGGGTGA
CNGC1 amino acid sequence (SEQ ID NO: 4)
MAKHQGPNNEIQMSITPDVSYDFHSPEALKPTREHGQPQASSKRWNWPAVAGVSSALNPWWNKILITSCVIAV
FFDPLFFYIPYTSEEKKCMGNDERVQTPALIFRSVTDIIFLVHIIYQLYVAIKFAHSRVSEDESLVFRYESVS
REQMIQFAKAFSHKLSWRSELTDVFAILPIPQLLIVGLFFNIGGNNYLLKRKIVGGFLLIQYVPRIYRIYLSS
VAVTESALWVRGVENFFLYILASHALGAFWYFCSIQRETSCWYRTCVATNISESKCTFYCHNSTAALITPQFK
TTLDEKCILKVPYNMTDPPFDFGIFFDALKNDIQGKINVPQKIGYCFWWGLRNLSNFGTSLETSTYLWENSFA
ILISIIGLLLFLYLIGNVQIYMQRATTRAEEVREKLRIKKNDIDNWVQNNGIEKDMKEEIMKNIVEKLENDIS
ADLDDIYSILPPYTRKVVKRFVGMRTLRNVPMLSQVDGRVLKMMCDYLKPVKYQENTMVFETGVPFDRMVFIT
DGSMWTYTIATDHQTSHSGKATSGGLVTSSSTEAHFLKKGDAYGHMLLQLASSSSTAVPISVANVRCHTKVEA
FVLMANDLRNIVTACGDLWPAFDYKASQDQAADQMAPPGHFQQQQGPKKRTVASSNIHPTGYSG*
CNGC2 cDNA sequence (SEQ ID NO: 5)
ATGGCTAAACATCAGGGACCAACCAATGAAATCCAAATGAGCATTACACCAGATGTGTCTTATGATTTCCATA
GTCCTGAGGCTCTAAAACCGACACCCGAACATGGACAACCTCAAGCAAGCAGCAAAAGACGGAATTGGCCAAC
AGTTGCCGGAGTTTCTTCAGCTTTGAACCCATGGTGGAATAAGATACTGGTAACTTCATGTGTGATTGCAGTT
TTATTTGATCCCTTGTTCTTTTACATTCCATACACCAGCGAGGAAAAAAAGTGTATGGGAAACGACGAAAGAG
TGCAGACTCCAGCTCTGATTTTCCGATCAGTCACAGACATCATTTTCCTGGTGCATATCATATACCAACTATA
TGTGGCTATCAAATTTGCACTCTCAAGGGTTTCCGAGGATGAAAGTTTGGTTTTTAGATATGAATCGGTATCC
CGAGAGCAAATGATTCAGTTTGCGAAGGCATTTTCTCACAAGTTGTCATGGCGATCTTTCCTAACTGACGTTT
TCGCTATTTTGCCCATACCACAACTGCTAATAGTAAGATTGTTTTTCAACATTGGAGGCAATAACTACTTGTT
AAAAAGAAAGATTGTGAGCGGTTTTCTTCTAATCCAATATGTGCCAAGAATCTATCGAATTTATCTTTCATCT
GTCGCGGTCACAGAATCTGCAATGTGGGTTAGAGGTGTATTTAACATTTTTCTCTACATTCTTGCGAGTCATG
CACTTGGAGCCTTTTGGTACTTTTGTTCTATTCAGCGAGAGACATTATGTTGGTATCGAACTTGCCTCGCTAC
AAATATATCAGAATCAAAATGCACCTTTTACTGCCATAACAGTGCTGCTGCACTTATGACCCCACAATTCAAG
ACAACTCTAGACGAGAAATGCATTCTAAAAGTTCCATATAACATGACCGATCCACCATATGATTTTGGAATAT
TTTTTGATGCCCTCAAAAATGATATCCAAGGGAAAATAAATGTTCCACAAAAGATTGGTTACTGTTTTTGGTG
GGGCTTGCGAAACTTGAGTAATTTTGGTACAAATTTGGAAACAAGTACCTATCTGTGGGAAAACAGCTTTGCC
ATTTTAATTTCTATCATCGGCTTGCTGTTGTTTTTATACCTCATTGGAAATGTACAGATTTATATGCAGAGGG
CAGTTACCAGAGCGGAGGAGGTAAGAGAGAAGCTTCGGATCAAAAAGAACGATATAGATAATTGGATCCAAAA
TAATGGTATCGAGAATGACATGAAGGAAGAGATCATGAAAAACATCGTTGAAAAGTTGGAAAACGACATAAGT
GCAGATCTGGATGATATATACTCTATTCTTCCCCCGTATACCAGGAAAGTTGTGAAGCGATTTGTCGGCATGA
GGACGCTAAGAAATGTACCAATGCTTAGCCAGGTGGATGGAAGAGTTTTGAAGATGATGTGCGACTATCTGAA
GCCGGTCAAGTACCAGGAGAATACAATGGTTTTTGAAACGGGAGTACCATTTGATAGAATGGTCTTCATTACA
GATGGGTCAATGTGGACTTACACGATTGCTACTGATCATCAGACTAGTCATTCTGGAAAAGCAACGTCAGGAG
GACTAGTTACTTCTTCGTCCACGGAAGCTCACTTCCTCAAGAAGGGTGATGCTTATGGACACATGCTTCTGCA
ATTAGCATCATCATCTTCTACGGCGGTTCCTATCTCAGTTGCAAATGTCAGGTGCCACACAAAGGTAGAGGCG
TTTGTCCTCATGGCCAATGACTTGAGAAACATAGTAACTGCGTGCGGAGATTTGTGGCCGGCTTTTGACTACA
AGGCTTCTCAAGATCAGGCGGCTGATCAGATGGCACCGCCGGGGCATTTTCAGCAACAACAAGGGCCAAAGAA
GCGTACTGTAGCATCATCAAATATCCATCCTACCGGCTACAGTGGGTGA
CNGC2 amino acid sequence (SEQ ID NO: 6)
MAKHQGPTNEIQMSITPDVSYDFHSPEALKPTPEHGQPQASSKRRNWPTVAGVSSALNPWWNKILVTSCVIAV
LFDPLFFYIPYTSEEKKCMGNDERVQTPALIFRSVTDIIFLVHIIYQLYVAIKFALSRVSEDESLVFRYESVS
REQMIQFAKAFSHKLSWRSFLTDVFAILPIPQLLIVRLFFNIGGNNYLLKRKIVSGELLIQYVPRIYRIYLSS
VAVTESAMWVRGVENIFLYILASHALGAFWYFCSIQRETLCWYRTCLATNISESKCTFYCHNSAAALMTPQFK
TTLDEKCILKVPYNMTDPPYDFGIFFDALKNDIQGKINVPQKIGYCFWWGLRNLSNFGTNLETSTYLWENSFA
ILISIIGLLLFLYLIGNVQIYMQRAVTRAEEVREKLRIKKNDIDNWIQNNGIENDMKEEIMKNIVEKLENDIS
ADLDDIYSILPPYTRKVVKRFVGMRTLRNVPMLSQVDGRVLKMMCDYLKPVKYQENTMVFETGVPFDRMVFIT
DGSMWTYTIATDHQTSHSGKATSGGLVTSSSTEAHFLKKGDAYGHMLLQLASSSSTAVPISVANVRCHTKVEA
FVLMANDLRNIVTACGDLWPAFDYKASQDQAADQMAPPGHFQQQQGPKKRTVASSNIHPTGYSG*

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

The following examples are provided to illustrate certain particular features and/or embodiments. These examples should not be construed to limit the disclosure to the particular features or embodiments described.

### Example 1. Fine-Mapping Analysis for FaRPc2

A total of 339 accessions, with three seasons of field phenotypic data from 2013-2015, were used for fine-mapping analysis. DNA of additional strawberry accessions was extracted by the simplified CTAB (cetyltrimethylammonium bromide) method with minor modifications (Keb-Llanes et al. 2002; Noh et al. 2017). To further fine-map the FaRPc2 region, we developed sub-genome specific high-resolution melting (HRM) markers by selecting FanaSNP array probes every 10 kb in the FaRPc2 region. We then selected HRM markers identifying H2 and H3 alleles through HRM analysis. The HRM markers representing 11 of Axiom® IStraw90 SNP probes and 10 of FanaSNP array probes encompassing the FaRPc2 region were used to genotype for 339 breeding germplasm.

PCR reactions of 5 μl contained 1 x Phire Hot Start II PCR buffer (Thermo Fisher Scientific, MA, USA), 0.2 mM dNTPs (New England BioLabs, MA, USA), 0.25 M of each forward and reverse primer, one unit of Phire Hot Start 1I DNA polymerase, 0.5×LCGreen Plus+ melting dye (BioFire, UT, USA), and 50 ng of DNA template. Amplification products were resolved by high-resolution melting analysis in LightCycler®480 system II (Roche Life Science, Germany). Melting curve data was analyzed by Melt Curve Genotyping and Gene Scanning software for Roche LightCycler 480 II system.

Utilizing map-based markers, the FaRPc2 locus was determined to be located in Chr. 7-3 of the recently published allo-octoploid strawberry genome. This FaRPc2 region comprises four predominant SNP haplotypes, namely, H1, H2, H3., and H4. Two haplotypes, H2 and H3, were found strongly linked with resistance. This region is responsible for about 25% phenotypic variation related to P. cactorum infection. Using 27 SNP markers. FaRPc2 was first mapped to low resolution and having the interval of 1.5 Mb on the distal end.

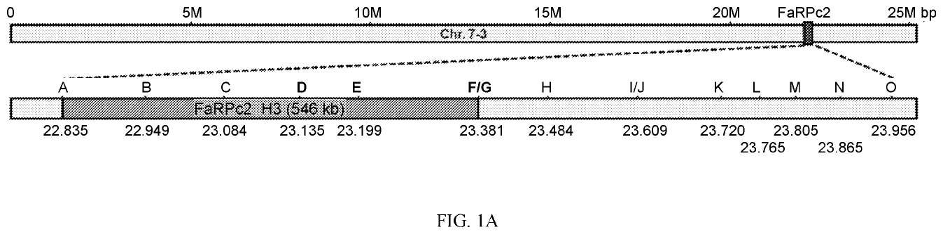

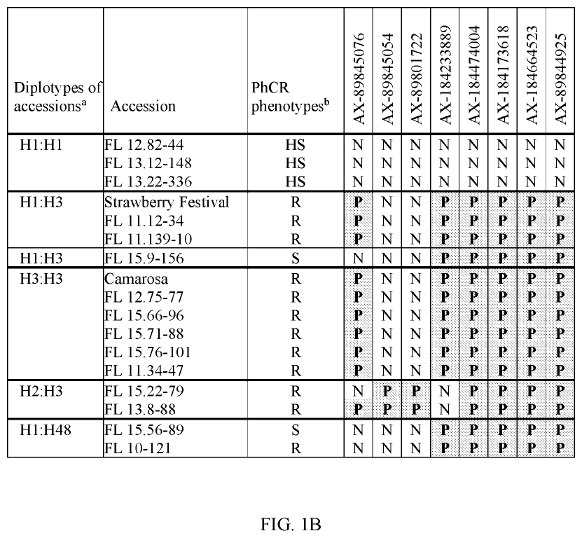

For the preliminary mapping of the FaRPc2 region, we mapped the FaRPc2 region using 11 markers derived from Axiom® k, IStraw90 SNP probes and 275 strawberry accessions from a previous study (Noh et al. 2018) with field phenotypic data for PhCR collected from three seasons, from 2012-2015 (FIG. 1A). To further fine map this 930 kb interval of the FaRPc2 region, a total of 339 strawberry accessions consisting of the 275 accessions above and 64 additional accessions, selected based on consistent field phenotypic data for PhCR collected from the three seasons of 2012-2015 were used. These accessions were identified to conduct family-based linkage studies and identify recombination within the family. In order to further resolve this interval, a total of eight sub-genome specific HRM markers, consisting of four H3 markers derived from previous study (Mangandi et al. 2017) and four HRM markers developed using the 50k FanaSNP array were added along with the previously used markers (FIG. 1A). In total, 339 accessions were genotyped with eight HRM markers and recombination events were identified for FaRPc2_H3 haplotype (FIG. 1A). In the FaRPc2_H3 region, it was confirmed that two recombinations occurred in the markers AX-89845076 and AX-184173618, respectively, allowing the FaRPc2_H3 region to be further narrowed from 930 kb to 546 kb (FIG. 1). The reduced FaRPc2 physical region for the FaRPc2 H3 haplotype was analyzed for the predicted genes using the haplotype-phased genomes A of FL 16.33-8. About 157 genes in H3 haplotype were annotated in the fine-mapped FaRPc2 region. Many genes related to plant pathogen defense, such as protein kinases and nucleotide-binding site (NBS)—leucine-rich repeat (LRR) proteins are located in this region.

### Example 2. Genome Sequencing, Assembly, and Genome Quality Analysis

Young leaves of FL 16.33-8 were selected as having the FaRPc2-H2 and H3 haplotype. For tissue etiolation for DNA extraction, FL 16.33-8 strawberry plants were covered with black plastic bags and kept in a greenhouse for 2 weeks. The etiolated young white leaf tissues were collected for DNA extraction. Genomic DNA extraction and genomic DNA libraries were prepared according to the manufacturer's instructions at DNA Link (Seoul, South Korea). The single-molecule real-time sequencing (SMRT) bell library was constructed using a PacBio DNA Template Prep Kit 1.0 (Pacific Biosciences). Quality and quantity of each library was checked using a 2100 Bioanalyzer (Agilent Technologies). The SMRT Bell-Polymerase complex was constructed using a PacBio Binding Kit 2.0 (Pacific Biosciences) based on the manufacturer's instructions. The complex was loaded onto SMRT cells (Pacific Biosciences, Sequel SMRT Cell 1M v2) and sequenced using Sequel Sequencing Kit 2.1 (Pacific Biosciences, Sequel SMRT Cell IM v2). For each SMRT cell, 1×600 min movies were captured using the Sequel sequencing platform (Pacific Biosciences) at DNA Link (Seoul, South Korea). The quality of Hifi was measured with LongQC (Fukasawa et al 2020). The genome of FL 16.33-8 was assembled using hifiasm, which is a de novo assembler that utilizes long, high-fidelity sequence reads to represent the haplotype information in a phased assembly graph. Two parents' Illumina paired-end (PE) data was utilized to phase haplotype scaffolds. The de novo genome assembly and raw genomic reads were used as input data for Ragtag, a tool designed for contig-ordering and scaffolding genome assemblies. Genome quality and completeness was assessed using Merqury ver 1.3, which evaluates assembly based on efficient k-mer set operations. The collinearity with the genetic linkage map for FL 16.33-8 and ‘Florida Brilliance’ was visually inspected using Circos. The macrosynteny with other strawberry diploid progenitors was inspected after identifying syntenic gene pairs using DAGChainer using SynMap within CoGe.

To identify and classify repetitive elements in the genome, LTR retrotransposon candidates were searched using LTRharvest and LTR_Finder and further identified and classified (e.g., Copia and Gypsy) using LTR_retriever. For the gene annotation, two haplotype-phased assemblies were annotated using Genome Sequence Annotation Server (GenSAS). Two methodologies were used in parallel to mask and annotate the repeat library. A library of predicted repeats detected with RepeatModeler (ISB RepeatModeler version 2.0.1) was combined with a library of repeats identified using RepeatMasker (ISB RepeatMasker version 4.1.1) and an Arabidopsis thaliana repeat library within GenSAS to create a consensus library of repeats and mask polished assembly. Nucleotide sequences were aligned to NCBI refseq plant using BlastN (version 2.7.1) as well as Diamond. These alignments were combined with results of several gene prediction models including AGUSTUS ver 3.3.1 to generate an official gene set and identify predicted transcripts within each masked assembly (EVidenceModeler, release Jun. 25, 2012). The whole set of genes were searched against agustus odb ver 10 using BUSCO ver 4.1.4. Genes were annotated with pfam domains using InterProScan (InterProScan, RRID:SCR 005829) v5.26-65.0.

The genome continuity of the Illumina draft genome assemblies was assessed with QUAST. A BUSCO analysis was performed using default parameters to assess the core gene set. The analysis was performed for genome assembly for FL 16.33-8. Whole-genome repetitive sequence and intact LTR retrotransposons were identified by EDTA (https://github.com/oushujun/EDTA). The LTR Assembly Index for each haplotype-phased genome was calculated based on the custom repeat library. Assembly consensus quality value (QV) was measured using k=21 with Merqury.

Haplotype-phased genome assembly and annotation of the resistance accession ‘FL 16.33-8’.

Strawberry accession FL 16.33-8 was selected as the reference genotype for whole-genome sequencing and assembly. To generate long-read assemblies, high-molecular weight DNA was isolated from the leaf tissue of FL 16.33-8 and used to create a high-quality haplotype-phased reference genome. The Sequel II generated a total of 637 Gb data with an average read length of 16,469 bp and produced ˜50.7 Gb HiFi reads with an average length of 18,925 bp.

The PacBio assembly showed higher contiguity, with a contig number of 1,468 in haplotype A and 592 in haplotype B, totaling 869.4 Mb and 826.9 Mb of total length and having a contig N50 of 16.4 Mb and 14.3 Mb in haplotype A and B, respectively (Table 2).

TABLE 2
Statistics on assembled FL 16.33-8 genome.

| | Haplotype | Haplotype |
|---|---|---|
| | A | B |
| Number of contigs (≥0 bp) | 1,468 | 592 |
| Number of contigs (≥1,000 bp) | 1,468 | 592 |
| Number of contigs (≥5,000 bp) | 1,468 | 592 |
| Number of contigs (≥10,000 bp) | 1,468 | 592 |
| Number of contigs (≥25,000 bp) | 1,447 | 587 |
| Number of contigs (≥50,000 bp) | 637 | 456 |
| Total length (≥0 bp) | 869,351,047 | 826,921,887 |
| Total length (≥1,000 bp) | 869,351,047 | 826,921,887 |
| Total length (≥5,000 bp) | 869,351,047 | 826,921,887 |
| Total length (≥10,000 bp) | 869,351,047 | 826,921,887 |
| Total length (≥25,000 bp) | 868,872,493 | 826,804,304 |
| Total length (≥50,000 bp) | 837,337,314 | 821,510,393 |
| Number of contigs | 1,468 | 592 |
| Largest contig | 27,317,239 | 27,253,556 |
| Total length | 869,351,047 | 826,921,887 |
| GC (%) | 40.1 | 39.99 |
| N50 | 16,418,230 | 14,284,693 |
| N75 | 10,170,993 | 9,465,336 |
| L50 | 23 | 22 |
| L75 | 39 | 40 |
| Number of N's per 100 kbp | 0 | 0 |

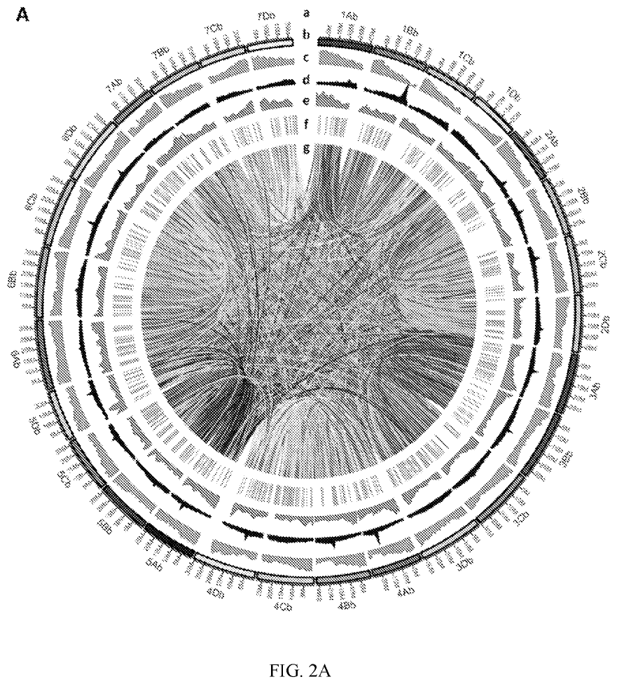

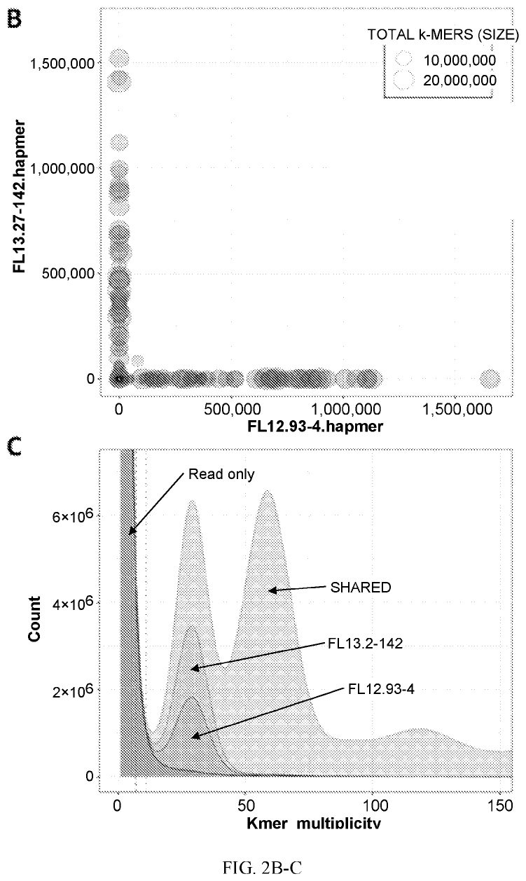

To achieve sub-chromosome-scale scaffolding, these data were assembled and scaffolded using the hifiasm (v0.12, NRGene, Nes Ziona, Israel). Trio binning genome assembly with hifiasm successfully classified both haplotypes of FL 16.33-8 genome, resulting in k-mer distributions for the read bins and an assembly that fully resolved both parental haplotypes (FIG. 2). In the k-mer blob plot of FL 16.33-8, there is good separation between the parental haplotypes, with each assembly consisting mostly of k-mers associated with the associated parental k-mer (FIG. 2B). This was also confirmed by the spectra plot for the combined assembly, where homozygous regions consist mostly of 2-copy k-mers and heterozygous regions consist mostly of 1-copy k-mers, as expected from the presence of both complete, parental haplotypes and low artefactual duplication (FIG. 2C).

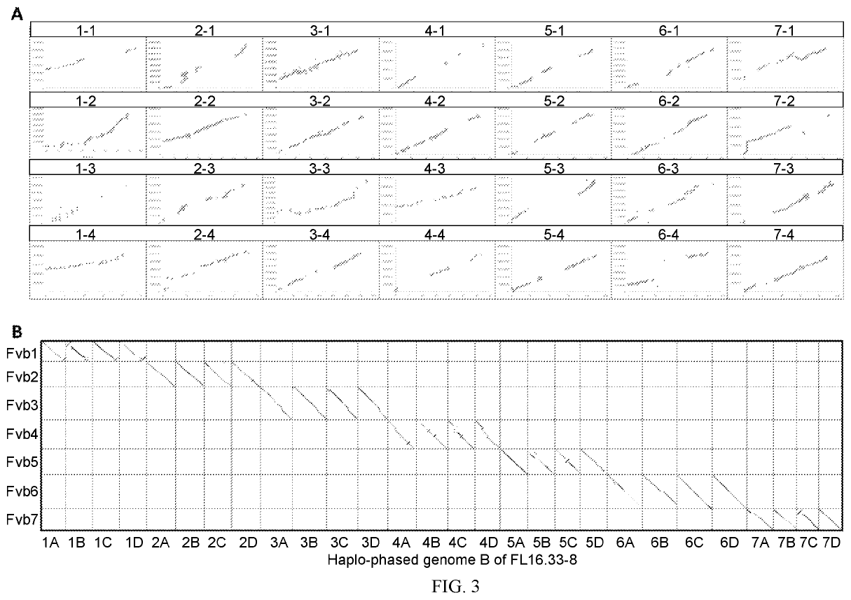

The total length of the final assembly is 782.4 Mb with 81 contigs in haplotype A and 778.1 Mb with 89 contigs in haplotype B distributed across 56 sub-chromosome-level pseudomolecules (FIG. 3A). The genome assembly allowed the identification of 56 sub-chromosomal pseudomolecules, and these pseudomolecules consisted of each of 28 haplotype A and B that were numbered and oriented according to the convention of reference genome ‘Camarosa’ (FIG. 3A). The shortest and longest chromosomes were Chr. 7-4 (19.35 Mb) and Chr. 6-3 (43.16 Mb) in haplotype A and Chr. 7-4 (22.11 Mb) and Chr. 6-2 (34.25 Mb) in haplotype B, respectively. The genome assembly and annotation completeness were assessed using the BUSCO software (Simao et al., 2015). The BUSCO results showed that 98.2% of complete BUSCO were detected in haplotype A and B of FL 16.33-8 assembly, including 0.12 and 0.24% of fragment BUSCO, and 1.67 and 1.61% of missing BUSCO, respectively. Overall, these metrics provided confirmation that we had obtained a high-quality genome sequence for the following functional investigations.

Transposable elements (TEs) were annotated in haplotype phased genomes A and B of FL 16.33-8 (FL 16.33-8 hap A and B) with EDTA pipeline (FIG. 2A). A total of 686.779 and 625,352 TEs within nine super-families were identified in FL 16.33-8 hap A and B, respectively. The total content of TE annotation of FL 16.33-8_hap A and B were 345 Mb and 343 Mb, including long terminal repeat (LTR) retrotransposons (Class I: 55.95% and 58.53%) and terminal inverted repeat (TIR) elements (Class II: 31.59% and 27.02%), respectively. The TEs were well distributed on the 28 chromosomes of FL 16.33-8 with a small increase in content near the centromeres (FIG. 2A). The most abundant Class I TEs were LTR retrotransposons, specifically the superfamily Gypsy followed by Copia, while for Class II transposons, Mutator was the most abundant. When comparing TEs in both haplotype phased genomes A and B of FL 16.33-8, we found that there was a difference between each super-family, and this analysis showed a high level of divergence in TE families between both haplotype phased genomes, resulting in structural and sequential variations in protein coding genes and their regulatory sequences. The quality of the genome was further assessed by examining the assembly continuity of repeat space using the LTR Assembly Index (LAI) deployed in the LTR_retriever package (v1.8).

The adjusted LAI score of haplotype phased genomes A and B of FL 16.33-8 was 15 and 14, respectively. Based on the LAI classification, this score is within the range of “reference” standard. Estimation of the regional LAI in 1 Mb sliding windows also showed that assembly continuity is uniform and of high quality across the entire genome (FIG. 2A).

TABLE 3
Classification and distribution of repetitive DNA elements identified
in the genome assembly for FL 16.33-8 by EDTA pipeline.

| | | | | | Length of masked | | Proportion of masked | |
|---|---|---|---|---|---|---|---|---|
| | | | Count | | sequence (bp) | | sequence (%) | |
| | Class | Sub-Class | Phased-1 | Phased-2 | Phased-1 | Phased-2 | Phased-1 | Phased-2 |
| LTR | Class I | | | | | | | |
| | | Copia | 49,819 | 46,878 | 36,018,090 | 36,449,119 | 4.14 | 4.41 |
| | | Gypsy | 78,855 | 80,419 | 88,250,218 | 94,067,744 | 10.15 | 11.38 |
| | | unknown | 125,213 | 124,478 | 68,541,731 | 69,967,337 | 7.88 | 8.46 |
| TIR | Class II | | | | | | | |
| | | CACTA | 96,289 | 58,887 | 41,265,249 | 25,374,277 | 4.75 | 3.07 |
| | | Mutator | 156,283 | 135,675 | 39,367,613 | 37,016,084 | 4.53 | 4.48 |
| | | PIF_Harbinger | 23,652 | 25,499 | 8,862,270 | 10,118,847 | 1.02 | 1.22 |
| | | Tc1_Mariner | 3,602 | 3,071 | 1,179,932 | 1,112,978 | 0.14 | 0.13 |
| | | hAT | 45,502 | 42,588 | 18,169,409 | 18,918,070 | 2.09 | 2.29 |
| NonTIR | Class II | | | | | | | |
| | | helitron | 107,564 | 107,857 | 42,929,696 | 49,479,243 | 4.94 | 5.98 |
| Total | — | — | 686,779 | 625,352 | 344,584,208 | 342,503,699 | 39.64 | 41.42 |

To validate haplotype phased genome assembly based on the final assembly, we used a high-density genetic linkage map developed by crossing ‘Florida Brilliance’ and FL 16.33-8. A total of 96 accessions were genotyped using FanaSNP with 50,000 subgenome specific SNPs and analyzed using JoinMap 4.0 software. The genetic map consisted of 13,910 SNP markers on 10,269 distinct positions in 31 LGs with a total genetic length of 2,382.03 cM. The genetic length of each LG ranged from 17.88 cM (LG3-3) to 166.84 cM (LG6-1) with an average length of 76.84 cM.

To compare the collinearity between the genetic linkage map and the newly assembled genome, marker sequences from the genetic linkage map were compared to the haplotype phased genome A of FL 16.33-8 (FIG. 3A). Using BLAST searches, a total of 40,371 FanaSNP showed a unique match to the FL 16.33-8 genome. A total of 31 LGs, from LG1-1 to LG7-4, were syntenic with one of the 28 sub-chromosomal pseudomolecules from the assembly. Less than 0.5% of markers mapped onto linkage groups conflicted with their position in the assembled chromosomes (FIG. 3A). The sub-genome specific FanaSNPs used in the construction of genetic linkage map were anchored to sub-chromosomal pseudomolecules of the reference genome and assigned to each sub-genome from Chr. 1-1 to Chr. 7-4 (FIG. 3A).

To gain further insight into the polyploid history of Fragaria x ananassa, we calculated the sequence similarity of each of the sub-chromosomal pseudomolecules by comparing the sequences of possible progenitors of this strawberry cultivar: F. vesca. F. iinumae, and F. virdis (FIG. 3B). In FIG. 3B, it is confirmed that the synteny block is conserved between F. vesca and FL 16.33-8 (F. ananassa) genome. The haplotype phased genome of FL 16.33-8 maintained a high level of collinearity with each of the three ancestral diploid strawberry genomes (FIG. 3B).

### Example 3. Comparative Genome Structure Analysis of FaRPc2 Regions

For the comparative genomic analysis of two different haplotypes of FaRPc2, H1 and H3, about 546 kb of the FaRPc2-H3 region identified with markers used for fine mapping were selected from several different haplotypes, including ‘Royal Royce’ (H1:H3), ‘Brilliance’ (H1:H1), and haplotype A of FL 16.33-8 (H3). Genomic sequences were compared with Mauve software in the Geneious Prime software 2020.2.4. The nucleotide and amino acid sequences of candidate genes, WAK and CNGC, were compared using MAFFT alignment in Geneious Prime software version 2020.2.4.

Comparative genomic analysis of FaRPc2-H3 region in octoploid reference genomes, ‘Brilliance’, FL 16.33-8, ‘Royal Royce’.

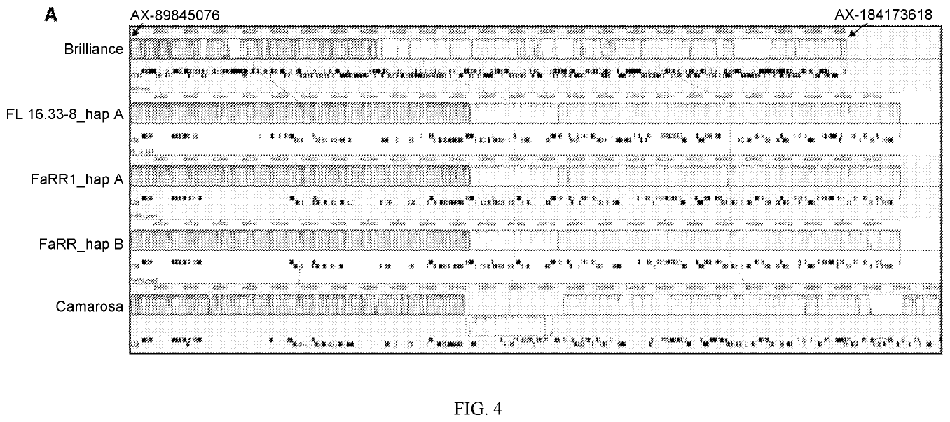

To identify genomic structure differences between various FaRPc2 H1 and H3 regions, we compared four genetic regions of FaRPc2-H3 on chromosome 7-3. About 546 kb of the FaRPc2-H3 region, identified with markers used for fine mapping, was selected from three different accessions: ‘Florida Brilliance,’ FL 16.33-8 hap A, ‘Royal Royce’ hap A and B, and ‘Camarosa,’ and the genome sequences were compared to using Mauve alignment program (FIG. 4). In the 546 kb region between the two HRM markers, AX-89845076 and AX-184173618, the H1 and H3 genotypes showed significant structure variations (SVs). However, the H3 genotypes in FL 16.33-8, “Royal Royce,’ and ‘Camarosa’ showed similar genomic structures in most regions. It was further confirmed that the nucleotide sequence was inverted in the about 230-285 kb region of ‘Camarosa’, an unphased genome. In addition, the UCD cultivars ‘Royal Royce’ and ‘Camarosa’ showed their own distinct genetic structures compared to the UF accession FL 16.33-8 with insertions and SNPs.

### Example 4. Identification of CNGC and WAK Genes Functionally Associated with FaRPc2-Mediated Resistance

Transcriptome analysis. Plants were grown for four weeks in a growth chamber under ideal conditions (73±2° F., Relative humidity 57-63%, 16 h light and 8 h darkness). A total of four breeding accessions were used for the RNA sequencing study: FL 11.28-34, FL 12.75-77, FL 12.82-44, and FL 13.22-336. All accessions were highly connected and comprised four haplotypes (H1-H4), as each of them has arose from an elite breeding population and had undergone repeated selection for >15 generations. The accession FL 11.28-34 is an H3:H1; heterozygous resistant haplotype. FL 12.75-77 is an H3:H3; homozygous resistant haplotype, FL 12.82-44 is an H1:H1: homozygous susceptible haplotype, and FL 13.22-336 is an H1:H1: homozygous susceptible haplotype against P. cactorum. All accessions were planted as plugs in sterile composite soil. The inoculation was prepared according to the method as exactly described previously (Mangandi et al. 2017). Crown tissues were collected at 0, 24, 48, 72, and 96 hours post inoculation (hpi) with at least three biological replicates.

For RNA extraction, crown tissues from each of the five time points were collected and samples were ground into fine powder using a mortar and pestle after flash freezing the tissue with liquid nitrogen. Total RNA was extracted according to the protocol of the Spectrum™ Plant Total RNA Kit (Sigma-Aldrich, St. Louis, MO, United States). To remove any traces of DNA, the isolated RNA was treated with DNAse I (Invitrogen) and resuspended in a total volume of 50 μl of RNase-free water. A total of 1 g total RNA was used for first-strand cDNA synthesis using the Transcriptor First Strand cDNA synthesis kit (Roche, Switzerland) according to the manufacturer's instructions. The RNA samples at times 0 and 96 hpi were selected for the RNA-seq study.

For the RNA-seq experiment, each of 0 and 96 hpi samples, for both water inoculated (WI) and pathogen inoculated (PI) groups, were sent to the Noble Research Institute (Ardmore, USA) for cDNA library preparation and sequencing. The paired-end library construction and sequencing was accomplished following standard Illumina sequencing methods using the TruSeq Stranded mRNA Library Prep Kit. The cDNA library was sequenced on the Nextseq 500 Illumina sequencing platform.

Illumina adapters were removed from the raw reads using Trimmomatic/0.33. After quality assessment with FastQC (Babraham Bioinformatics), the filtered reads were then mapped to each haplotype phased genome assembly using HISAT2. The sequence alignment map (SAM) format was converted to Binary Alignment Map (BAM) format. To import the BAM files into R, ‘Rsamtools’ package was used. Differentially expressed genes in resistant vs susceptible were identified using the DESeq2 pipeline. DESeq2 software was employed to calculate the expression between genotypes (Wang et al., 2010).

Pairwise comparison was performed between H1-WI versus H1-PI, H1-PI versus H3-PI, H3-WI versus H3-PI, and total resistance versus total susceptibility. The genes with a log 2 fold change ≥1 and FDR-adjusted p-value (q-value)<0.05 were selected as differentially expressed genes (DEGs). Heatmap was generated using R package ‘gplots’ using expression data for genes locating at the interval of FaRPc2.

The differentially expressed sequences were annotated with the help of a sequence homology search using blastx program in the BLAST+ package (Camacho et al. 2009). The sequences were analyzed at an e-value of 1e-6 to the NCBI non-redundant protein database. The homologues were selected on the basis of the e-value of less than 1e-6 and the highest high-scoring segment pairs (HSP).

The functions of differentially expressed genes were characterized using the software HMMER2GO (https://github.com/sestaton/HMMER2GO) by using Gene Ontology (GO) terms. The enrichment pattern of different gene ontology process were performed with PGSEA (Furge and Dykema 2006). The differentially expressed sequences pathway annotation at Kyoto Encyclopedia of Genes and Genomes was accomplished by KEGG Automatic Annotation Server (Moriya et al. 2007) implementing the single directional best hit method. The pathway map was visualized with KeggExp (Liu et al. 2019).

In this study, the result of fine mapping was integrated with RNA-Seq reads from four accessions at 96 hpi following pathogen infestation, comprising accessions either susceptible or resistant against P. cactorum. The average GC content for the total reads was about 46%, which pointed to F. x ananassa transcriptome data having more AT content and is in line with other non-model plant transcriptome sequences. More than 88% of clean sequencing reads were mapped to the reference coding DNA sequence (CDS) of octoploid cultivated F. x ananassa. About 3,040 differentially expressed genes were explored, with numerous differentially expressed genes possibly involved in imparting PhCR resistance, and thus, providing a basis for further study. The transcripts mapped to reference CDS allowed for a sense of whole genome measurement of transcript abundance and provided important information about transcriptional and post-transcriptional modulation. Our purpose was to characterize the differential expression in octoploid strawberry for the transcriptomes of different haplotypes in general, so we concentrated on DEGs that expressed in common or were haplotype-specific in response to P. cactorum. Haplotype H3 accessions, known to be resistant from elite breeding, provided a concrete basis for deciphering genes having role in defense against P. cactorum. The differentially expressed CDS showed high homology with members of rosaceae family, and F. vesca shared the highest number of homology with our data. The genome of F. vesca shares high sequence similarity with the octoploid F. x ananassa. Therefore, it is not surprising that our RNA-Seq data shows highest homology with F. vesca.

To identify critical genes involved in F. x ananassa resistance against the infection of P. cactorum, eight Illumina libraries were constructed. Further, for a broad representation of the compatible and incompatible interaction, we pooled H3 haplotype resistant individuals (FL 12.75-77 and FL 11.28-34) into one group, H3, and compared with pooled H1 haplotype susceptible individuals (FL 12.82-44 and FL 13.22-336). The isolate 12-420 of P. cactorum was found to be highly virulent and to our knowledge there is no race or pathotype present for P. cactorum. We chose a 96 hpi sampling time to examine for significant alterations in gene expression.

A total of 389,997,518 raw reads of Illumina sequence was produced from the RNA-Seq experiment and the number of raw reads in each library ranged from 41.15 to 55.97 million paired end reads (average 101 bp). After trimming more than 97.8% paired-end reads through quality filtering, more than 91% of those were mapped to CDS of F. x ananassa. To analyze the expression of the genes between resistant (FaRPc2-H3) and susceptible (FaRPc2-H1), gene expression analysis was performed by using the CDS of haplotype phased genome A of FL 16.33-8 as a reference.

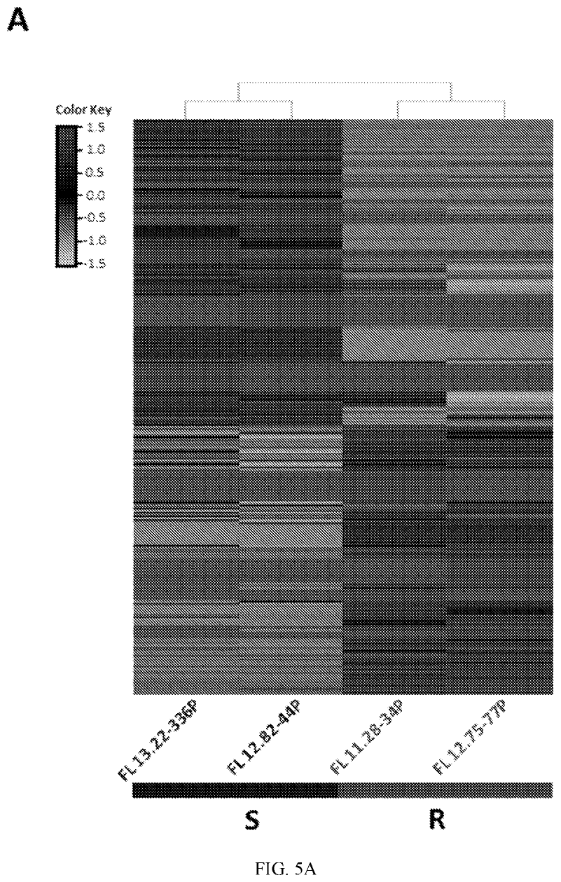

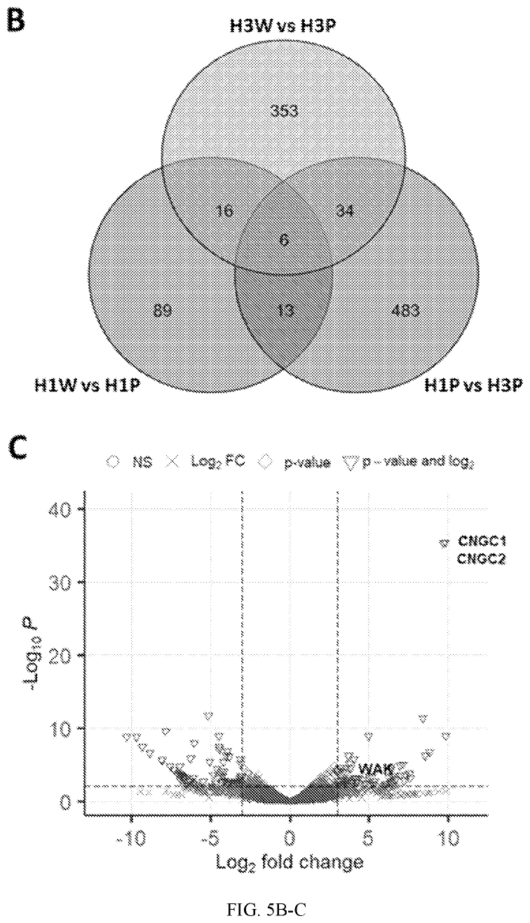

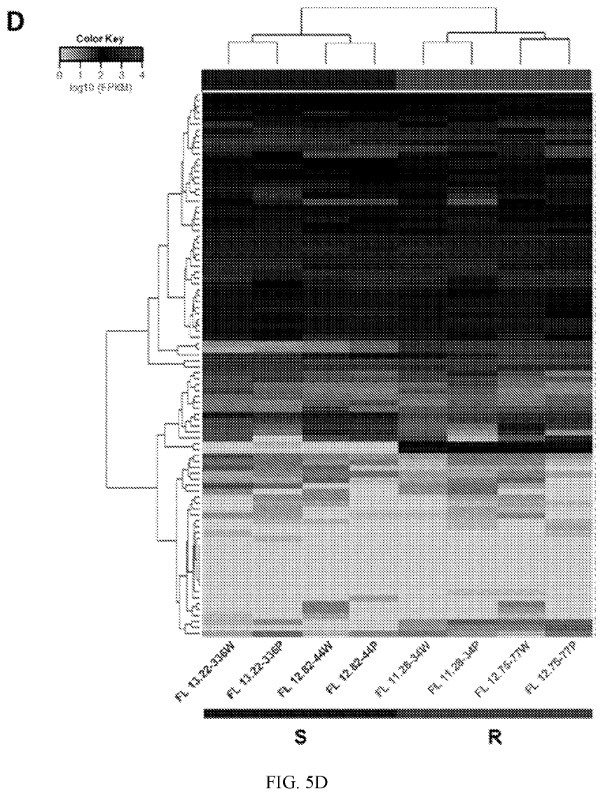

The genome wide heatmap visualization of these DEGs at the whole-genome level showed the significant patterns of differential gene expression between susceptible (H1) and resistant (H3) accessions (FIG. 5A). The hierarchical clustering shows two groups of differentially expressed genes in response to the pathogen infection with P. cactorum between resistant (H3) and susceptible (H1) haplotypes (FIG. 5A). This finding indicates that there is a specific group of genes expressed in resistant haplotypes for the defense pathway against P. cactorum.

The Venn diagram analysis was carried out to identify overlap of upregulated DEGs in the water and PI treatments between FaRPc2-H1 and H3 genotypes (FIG. 5B). In the comparison of FaPc2-H1 and H3 haplotypes with haplotype phased genome A, a total of 409 genes were upregulated between H3-WI and H3-PI treatment, whereas 124 genes were upregulated between H1-WI and H1-PI treatment. A total of 536 DEGs were upregulated between H1-PI and H3-PI treatment. The total of 6 DEGs were common in all comparisons. The large number of genes related to plant defense responses were highly upregulated in the resistant compared to the susceptible accession (FIG. 5B). Differentially expressed genes between H1-PI and H3-PI were visualized using a volcano plot (FIG. 5C). Volcano plot analysis of DEGs with whole genome levels showed remarkably differentially expressed genes; namely, two cyclic nucleotide-gated ion channel 1-like (CNGCs). The CDS were assigned GO processes that are further subcategorized into Cellular Component (CC), Molecular Function (MF), and Biological Process (BP). In the comparison of H1-PI versus H3-PI, catalytic activity and binding were the most represented GO terms in the MF category. In the BP category, metabolic processes and cellular processes were the most conspicuous GO category in the comparison of H1-PI versus H3-PI.

By narrowing the major locus FaRPc2-H3 with the help of fine-mapping, we were able to select a relatively smaller number of candidate genes. To identify expression changes of genes in response to P. cactorum infection in the FaRPc2-H3 region, a heatmap was created comprising the genes showing expression changes in the 546 kb of the fine mapped FaRPc2-H3 region (FIG. 4 and FIG. 5D). Concentrating only at the FaRPc2-H3 region, a total of 92 genes showed a difference in expression between the susceptible and resistant accessions in response to P. cactorum (FIG. 5D).

In the FaRPc2-H3 region, expression of two gene families in particular—cyclic nucleotide-gated ion channel 1-like (CNGCs) and wall-associated receptor kinase 1-like (WAK1)—were higher in all resistant haplotypes as compared to susceptible haplotypes. Two other genes—protein_DEFECTIVE_IN_MERISTEM_SILENCING 3-like, signal_recognitionparticle_54_kDa_protein_2-were also upregulated in the FaRPc2-H3 region (FIG. 5D). Two CNGCs, which represented two of the four genes most highly expressed genes in the FaRPc2-H3 region, also showed the highest expression in volcano plots using DEGs at the whole genome level. In addition, the transcriptome analysis of FaRPc2-H3 region indicated the upregulation of WAK1 against P. cactorum in the resistance accessions. Taken together, the candidate genes WAK1 and two CNGCs (CNGC1 and CNGC2), in the FaRPc2-H3 regions were predicted to influence resistance against P. cactorum.

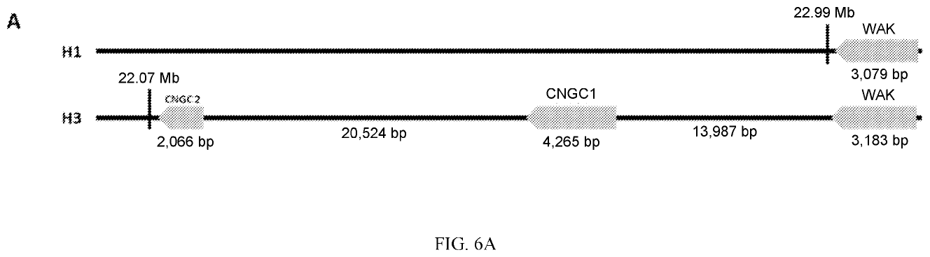

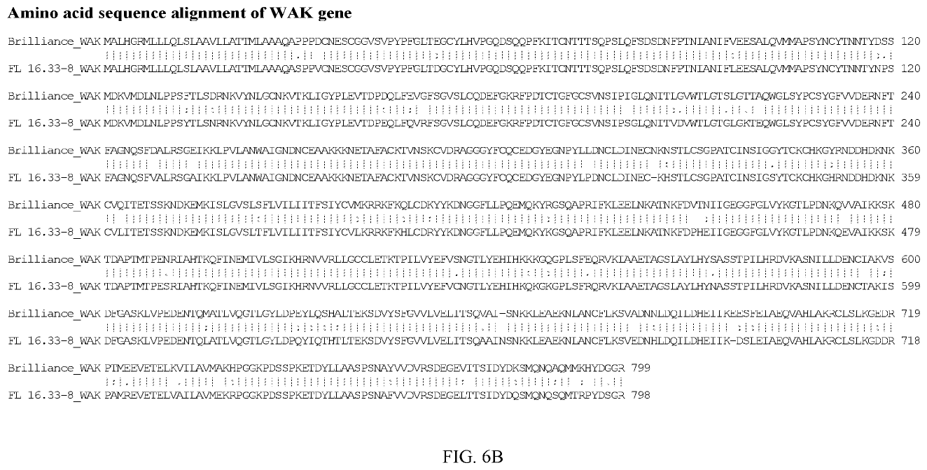

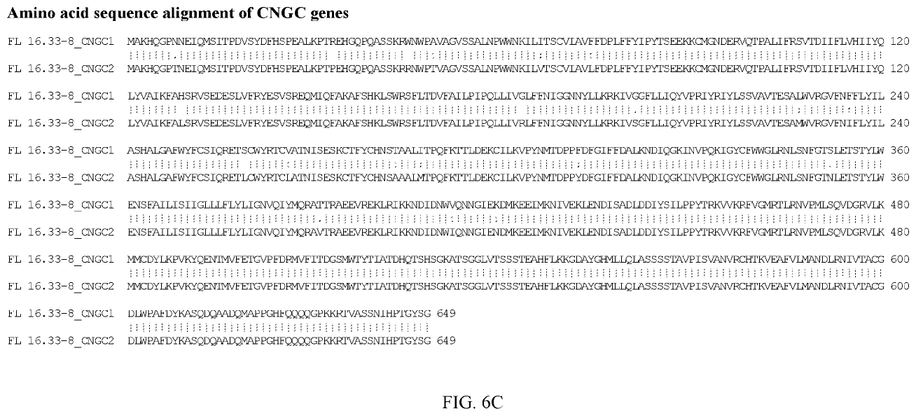

When we compared the FaRPc2-H3 regions of ‘Florida Brilliance’ and FL 16.33-8_Hap A to identify candidate genes between susceptible (H1) and resistant (H3) haplotypes. ‘Florida Brilliance’ and FL 16.33-8 Hap A each had one WAK, and only FL 16.33-8_Hap A had two CNGCs (FIG. 6A). The predicted nucleotide and amino acid sequences of WAK and CNGC genes from FL 16.33-8_Hap A were aligned and compared with resistance genotype ‘Royal Royce’ and susceptibility genotype ‘Florida Brilliance’ (FIG. 6B). As a result of amino acid sequence alignment with three accessions, WAK was 100% consistent with ‘Royal Royce’, and 91.25% consistent with ‘Florida Brilliance’, which is the susceptibility haplotype (FIG. 6B). In the amino acid sequence alignment result of the two CNGC genes of FL16.33-8_Hap A and ‘Royal Royce’. CNGC1 of FL 16.33-8 was 100% identical to CNGC1 of ‘Royal Royce’ Hap A and Hap B, and CNGC2 of FL 16.33-8 was 100% identical to CNGC2 of ‘Royal Royce’ hap1 and hap2. The two CNGC1 and CNGC2 genes were consistent at 98.67% (FIG. 6B).

Thus, the two promising candidate genes based on RNA-Seq analysis in the FaRPc2-H3 region were WAK and CNGCs, and the corresponding open reading frames (ORFs) were extracted. The conserved domain search showed high structural similarity and domain architecture in the NCBI database. The WAK sequence N-terminal region showed homology with extracellular domain containing cysteine-rich galacturonan-binding domain (GUB-WAK_bind; pfam13947) which is the extracellular portion of a serine/threonine kinase that binds to the cell-wall pectin. This cysteine-rich domain is characteristic of WAK. The GUB-WAK_bind domain is further followed by the catalytic domain of the serine/threonine kinases (STKc_IRAK; cd14066) which is part of a larger superfamily (PKc like superfamily) that comprises protein kinases. This subfamily includes plant receptor-like kinases (RLKs) involved in plant resistance to pathogen infection. Additionally, there is a calcium-binding EGF-like domain (EGF_CA; smart00179) between the GUB-WAK_bind and STKc_IRAK domains. In the CNGC sequence, there is the presence of CAP ED super-family domain (cd00047), which is the effector domain for the cAMP receptor protein family of transcription factors. This domain aids in the binding of effectors and initiates conformational change ultimately leading to activation of transcription.

### Example 5. Transient Gene Silencing of FaWAK and FaCNGC in Strawberry

Root transient RNAi expression in strawberries by A. tumefaciens was performed according to a modified Zhong method (Zhong et al. 2016). We used octoploid strawberry (Fragaria x ananassa Duchesne) cultivars ‘Florida Beauty and ‘Fronteras’ in this study. Also, we used vegetative generation of Florida Beauty and ‘Fronteras’ plants, which were propagated from runner cutting and grown in a greenhouse at 18° C.-28° C. and under mist with a 16-hour light/8-hour dark light cycle for 3 weeks. After 3 weeks, the runner propagation plants were transferred to the growth room at 23° C. with 12-hour light/12-hour dark light cycle conditions for the transient gene expression study.

Three (3) week-old strawberry plants were used to perform transient expression analysis and to confirm FaRPc2 resistance in strawberry root and crown. We used the pK7GWIWG(II) vector system for silencing FaWAK and FaCNGC genes for the transient expression assay. The partial sequence of 330 bp FaWAK and 356 bp FaCNGC genes were cloned into the pK7GWIWG(II) RNAi silencing vector in sense and antisense orientation site by an intron using the GATEWAY cloning system. The final product of the generated pK7::FaWAK and pk7::FaCNGC silencing vectors were introduced into A. tumefacient EHA105 using the electroporation method. A, tumefacient was grown at 28° C. overnight in LB medium with rifampicin (50 mg/L) and spectinomycin (50 mg/L). When the culture density reached an OD₆₀₀ of 1.0, A, tumefacient were harvested by centrifuge at 4500 rpm for 25 min and resuspended and adjusted to an OD₆₀₀ of 0.6 in MMA activation buffer (10 mM 2-(N-morpholino) ethanesulphonic acid (MES), 10 mM MgCl₂, and 20 μM acetosyringone; pH5.6) and shaken for 3 hours at room temperature before infiltration of strawberry root and crown.

In order to increase the transformation efficiency, we used the vacuum infiltration method. Briefly, the roots and crowns were scraped with a syringe needle before vacuum infiltration and transferred to a suspension of A. tumefaciens with 0.05% silwet L-77. Vacuum infiltration was applied for 5 min. After infiltration, inoculated plants were transferred to a 4-inch pot in the humid box. Inoculated plants were cultured in a growth room (12/12 hr light/dark cycle, 23-25° C.). Each experiment was performed in biological triplicate for each treatment group, and empty pK7GWIWG(II) in A. tumefaciens was used as a control and treatment.

After inoculation, the root and crown were collected to confirm gene expression of FaWAK and FaCNGC. Total RNA was isolated from the crown to top of the root using the Spectrum™ Plant Total RNA Kit (Sigma-Aldrich, St. Louis, MO, United States) following the manufacturer's protocol. Subsequently, lug RNA was treated with DNase Amplification Grade Kit (Invitrogen) to eliminate DNA contamination. Then, cDNA synthesis was performed following the First Strand cDNA Synthesis Strand protocol (New England BioLabs. Ipswich, USA) using the oligo (dt) primer (IDT, Coralville, IA, USA). qRT-PCR was conducted with Forget-Me-Not qPCR master Mix Kit from Biotium.

1. A genetically modified Fragaria plant, wherein the genome of the Fragaria plant has been modified to express:
(a) a heterologous WAK1 gene, coding sequence, or fragment thereof, or a heterologous nucleic acid sequence encoding SEQ ID NO: 2;
(b) a heterologous CNGC1 gene, coding sequence, or fragment thereof, or a heterologous nucleic acid sequence encoding SEQ ID NO: 4; and/or
(c) a heterologous CNGC2 gene, coding sequence, or fragment thereof, or a heterologous nucleic acid sequence encoding SEQ ID NO: 6.

2. (canceled)

3. The genetically modified Fragaria plant of claim 2, wherein the Fragaria plant has been genetically modified to contain an insertion of:
(a) SEQ ID NOs: 1 and 3;
(b) SEQ ID NOs: 1 and 5;
(c) SEQ ID NOs: 3 and 5;
(d) SEQ ID NOs: 1, 3, and 5;
(e) the heterologous nucleic acid sequence encoding SEQ ID NO: 2 and the heterologous nucleic acid sequence encoding SEQ ID NO: 4;
(f) the heterologous nucleic acid sequence encoding SEQ ID NO: 2 and the heterologous nucleic acid sequence encoding SEQ ID NO: 6;
(g) the heterologous nucleic acid sequence encoding SEQ ID NO: 4 and the heterologous nucleic acid sequence encoding SEQ ID NO: 6; or
(h) the heterologous nucleic acid sequence encoding SEQ ID NO: 2, the heterologous nucleic acid sequence encoding SEQ ID NO: 4, and the heterologous nucleic acid sequence encoding SEQ ID NO: 6.

4. (canceled)

5. The genetically modified Fragaria plant of claim 1, wherein genetically modified Fragaria plant has increased resistance to Phytophthora crown rot (PhCR) disease caused by infestation with Phytophthora cactorum compared to a control Fragaria plant the doesn't express the heterologous WAK1 gene, coding sequence, or fragment thereof, the heterologous CNGC1 gene, coding sequence, or fragment thereof, or the heterologous CNGC2 gene, coding sequence, or fragment thereof.

6. A method of genetically modifying a Fragaria plant cell comprising introducing one or more of:
(a) a heterologous WAK1 gene, coding sequence, or fragment thereof, or a heterologous nucleic acid sequence encoding SEQ ID NO: 2;
(b) a heterologous CNGC1 gene, coding sequence, or fragment thereof, or a heterologous nucleic acid sequence encoding SEQ ID NO: 4; and/or
(c) a heterologous CNGC2 gene, coding sequence, or fragment thereof, or a heterologous nucleic acid sequence encoding SEQ ID NO: 6;
into the genome of the Fragaria plant cell through one or more of: unaided homologous recombination, recombinase-based insertion, and DNA repair-based insertion.

7. (canceled)

8. The method of claim 6, wherein the DNA repair-based insertion is mediated by a zinc finger nuclease (ZFN), a Transcription Activator-Like Effector Nuclease (TALEN), a meganuclease, or a Clustered Regularly Interspaced Short Palindromic Repeats (CRISPR)-associated (Cas) protein and a guide RNA (gRNA).

9. (canceled)

10. The method of claim 8, wherein:
(i) the Cas protein comprises a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease; and
(ii) the guide RNA comprises a CRISPR RNA (crRNA) and a trans-activating CRISPR RNA (tracrRNA), as separate molecules or as a single chimeric guide RNA (sgRNA).

11. (canceled)

12. The method of claim 6, wherein introducing:
(a) the heterologous WAK1 gene, coding sequence, or fragment thereof, or the heterologous nucleic acid sequence encoding SEQ ID NO: 2;
(b) the heterologous CNGC1 gene, coding sequence, or fragment thereof, or the heterologous nucleic acid sequence encoding SEQ ID NO: 4; and/or
(c) the heterologous CNGC2 gene, coding sequence, or fragment thereof, or the heterologous nucleic acid sequence encoding SEQ ID NO: 6;
into a Fragaria plant cell comprises electroporation, microprojectile bombardment, biolistic transformation, microinjection, protoplast transformation, an Agrobacterium tumefaciens vector transformation or an Agrobacterium rhizogenes vector transformation.

13. The method of claim 6, wherein the method further comprises generating one or more regenerants following introducing:
(a) the heterologous WAK1 gene, coding sequence, or fragment thereof, or the heterologous nucleic acid sequence encoding SEQ ID NO: 2;
(b) the heterologous CNGC1 gene, coding sequence, or fragment thereof, or the heterologous nucleic acid sequence encoding SEQ ID NO: 4; and/or
(c) the heterologous CNGC2 gene, coding sequence, or fragment thereof, or the heterologous nucleic acid sequence encoding SEQ ID NO: 6;
into the Fragaria plant cell.

14. (canceled)

15. The method of claim 6 wherein the method further comprises selecting one or more T₀ plants containing one or more of:
(a) the heterologous WAK1 gene, coding sequence, or fragment thereof, or the heterologous nucleic acid sequence encoding SEQ ID NO: 2;
(b) the heterologous CNGC1 gene, coding sequence, or fragment thereof, or the heterologous nucleic acid sequence encoding SEQ ID NO: 4; and
(c) the heterologous CNGC2 gene, coding sequence, or fragment thereof, or the heterologous nucleic acid sequence encoding SEQ ID NO: 6.

16. (canceled)

17. A method of genetically modifying a Fragaria plant, the method comprising: introducing a CRISPR system into a Fragaria plant cell, wherein the CRISPR system comprises:
(a) an RNA-guided DNA endonuclease or a nucleic acid encoding the RNA-guided DNA endonuclease:
(b) a guide RNA or a nucleic acid encoding the guide RNA; and
(c) a DNA donor template containing a sequence encoding one or more of:
(i) a heterologous WAK1 gene, coding sequence, or fragment thereof, or a heterologous nucleic acid sequence encoding SEQ ID NO: 2;
(ii) a heterologous CNGC1 gene, coding sequence, or fragment thereof, or a heterologous nucleic acid sequence encoding SEQ ID NO: 4; and/or
(iii) a heterologous CNGC2 gene, coding sequence, or fragment thereof, or a heterologous nucleic acid sequence encoding SEQ ID NO: 6;
wherein the RNA-guided DNA endonuclease and the guide RNA form a complex that creates a single or double strand break in the genome of the Fragaria plant cell and the DNA donor template is used by the plant cell to repair the break and insert the nucleic acid sequence encoding:
(i) the heterologous WAK1 gene, coding sequence, or fragment thereof, or the heterologous nucleic acid sequence encoding SEQ ID NO: 2;
(ii) the heterologous CNGC1 gene, coding sequence, or fragment thereof, or the heterologous nucleic acid sequence encoding SEQ ID NO: 4; and/or
(iii) the heterologous CNGC2 gene, coding sequence, or fragment thereof, or the heterologous nucleic acid sequence encoding SEQ ID NO: 6;
into the genome at the site of the single or double strand break.

18. The method of claim 17, wherein the CRISPR system is selected from the group consisting of: a CRISPR class 1 system, a CRISPR class 2 system, a CRISPR/Cas system, a CRISPR/Cas9 system, a CRISPR/zCas9 system and a CRISPR/Cas3 system.

19. (canceled)

20. (canceled)

21. The method of claim 17, wherein introducing a CRISPR system into a Fragaria plant cell comprises electroporation, microprojectile bombardment, biolistic transformation, microinjection, protoplast transformation, an Agrobacterium tumefaciens vector transformation or an Agrobacterium rhizogenes vector transformation.

22. The method of claim 17, wherein the DNA donor template comprises SEQ ID NO: 1 or a fragment thereof or a complement thereof, SEQ ID NO: 3 or a fragment thereof or a complement thereof, or SEQ ID NO: 5 or a fragment thereof or a complement thereof.

23. The method of claim 17, wherein CRISPR system targets the FaRPc2 locus.

24. The method of claim 17, wherein the genetically modified Fragaria plant cell overexpresses:
(i) the heterologous WAK1 gene, coding sequence, or fragment thereof, or the heterologous nucleic acid sequence encoding SEQ ID NO: 2;
(ii) the heterologous CNGC1 gene, coding sequence, or fragment thereof, or the heterologous nucleic acid sequence encoding SEQ ID NO: 4; and/or
(iii) the heterologous CNGC2 gene, coding sequence, or fragment thereof, or the heterologous nucleic acid sequence encoding SEQ ID NO: 6.

25. The method of claim 17, wherein the method further comprises generating one or more regenerants following introducing the CRISPR system into a Fragaria plant cell.

26. The method of claim 25, wherein the method further comprises analyzing one or more regenerants for expression of:
(i) the heterologous WAK1 gene, coding sequence, or fragment thereof, or the heterologous nucleic acid sequence encoding SEQ ID NO: 2;
(ii) the heterologous CNGC1 gene, coding sequence, or fragment thereof, or the heterologous nucleic acid sequence encoding SEQ ID NO: 4; and/or
(iii) the heterologous CNGC2 gene, coding sequence, or fragment thereof, or the heterologous nucleic acid sequence encoding SEQ ID NO: 6.

27. The method of claim 26, wherein the method further comprises selecting one or more T₀ plants expressing:
(i) the heterologous WAK1 gene, coding sequence, or fragment thereof, or the heterologous nucleic acid sequence encoding SEQ ID NO: 2;
(ii) the heterologous CNGC1 gene, coding sequence, or fragment thereof, or the heterologous nucleic acid sequence encoding SEQ ID NO: 4; and/or
(iii) the heterologous CNGC2 gene, coding sequence, or fragment thereof, or the heterologous nucleic acid sequence encoding SEQ ID NO: 6.

28. The method of claim 25, wherein the regenerant or the T₀ plant has increased resistance to Phytophthora crown rot disease.

29. (canceled)

30. (canceled)

31. (canceled)

32. (canceled)

33. (canceled)

34. The genetically modified Fragaria plant of claim 1, wherein the Fragaria plant is an octoploid Fragaria plant.
